(19) United States
(12) Patent Application Publication
(10) Pub. No.: US 2019/0105269 A1
(43) Pub. Date: Apr. 11, 2019

(54) COMPOSITIONS, METHODS, COMBINATION TREATMENTS

(71) Applicant: CMPD Licensing, LLC, Conroe, TX (US)

(72) Inventor: Jay Richard Ray, II, Conroe, TX (US)

(73) Assignee: CMPD Licensing, LLC, Conroe, TX (US)

(21) Appl. No.: 16/215,150

(22) Filed: Dec. 10, 2018

Related U.S. Application Data
(63) Continuation-in-part of application No. 15/976,579, filed on May 10, 2018.
Application No. 15/976,579 is a continuation-in-part of application No. 14/990,168, filed on Jan. 7, 2016.
Application No. 14/990,168 is a continuation-in-part of application No. 15/597,936, filed on May 17, 2017, now Pat. No. 10,105,342.
Application No. 15/597,936 is a continuation-in-part of application No. 15/668,184, filed on Aug. 3, 2017.
Application No. 15/597,936 is a continuation-in-part of application No. 15/440,800, filed on Feb. 23, 2017.
Application No. 15/440,800 is a continuation-in-part of application No. 14/975,172, filed on Dec. 18, 2015, now Pat. No. 9,707,229.
Application No. 14/975,172 is a continuation-in-part of application No. 14/819,342, filed on Aug. 5, 2015.
(60) Provisional application No. 62/298,994, filed on Feb. 23, 2016.
(60) Provisional application No. 62/298,991, filed on Feb. 23, 2016.
(60) Provisional application No. 62/370,571, filed on Aug. 3, 2016.

(51) Int. Cl. A61K 9/06; A61K 31/7036; A61K 31/351; A61K 31/496; A61K 31/17; A61K 38/12; A61K 31/506; A61K 31/65; A61K 31/4178; A61K 31/4174; A61K 47/20; A61K 31/196; A61K 9/48; A61K 9/20; A61K 9/14; A61P 31/10; A61P 31/04; A61K 9/00

(52) U.S. Cl. CPC A61K 9/06; A61K 9/0014; A61K 31/351; A61K 31/496; A61K 31/17; A61K 38/12; A61K 31/506; A61K 31/65; A61K 31/4178; A61K 31/4174; A61K 47/20; A61K 31/196; A61K 9/48; A61K 9/20; A61K 9/14; A61P 31/10; A61P 31/04; A61K 31/7036

(57) ABSTRACT

A combination treatment method for treating a bacterial or fungal nail infection may include providing one or more first composition components and one or more second composition component. The first and second composition components may be mixed with diluent to formulate respective first and second topical nail composition. The topical nail compositions may include nail lacquers for application to an infected nail in a thin layer. The one or more first composition components may include mupirocin. The one or more second composition components may include tetracycline, nitrofurantoin, or combination thereof.

### CROSS-REFERENCE TO RELATED APPLICATIONS

The present application is a continuation-in-part of U.S. patent application Ser. No. 15/976,579, filed May 10, 2018, which is hereby incorporated by reference. U.S. patent application Ser. No. 15/976,579 is a continuation-in-part of U.S. patent application Ser. No. 14/990,168, filed Jan. 7, 2016, U.S. patent application Ser. No. 15/597,936, filed May 17, 2017, and U.S. patent application Ser. No. 15/668,184, filed Aug. 3, 2017, each of which is hereby incorporated herein by reference. U.S. patent application Ser. No. 15/597,936 is a continuation-in-part application of U.S. patent application Ser. No. 15/440,800, filed Feb. 23, 2017, U.S. patent application Ser. No. 14/975,172, filed Dec. 18, 2015 (now U.S. Pat. No. 9,707,229), and U.S. patent application Ser. No. 14/819,342, filed Aug. 5, 2015, each of which is hereby incorporated herein by reference. U.S. patent application Ser. No. 15/440,800 claims priority under 35 U.S.C. § 119(e) to U.S. Provisional Patent Application No. 62/298,991, filed Feb. 23, 2016, and U.S. Provisional Patent Application No. 62/298,994, filed Feb. 23, 2016, each of which is hereby incorporated herein by reference. U.S. patent application Ser. No. 15/668,184 claims priority under 35 U.S.C. § 119(e) to U.S. Provisional Patent Application No. 62/370,571, filed on Aug. 3, 2016, which is hereby incorporated herein by reference.

### FIELD OF THE INVENTION

The present application relates to compounded compositions, methods of making compounded compositions, and methods of using compounded compositions to treat or prevent an infection. More specifically, the present application relates compounded topical nail compositions, methods of making compounded topical nail compositions, and methods of using compounded topical nail compositions to treat nail infections.

### BACKGROUND

The body normally serves as host for a variety of bacteria and fungi. Most of the time, the balance between the body as host and the microorganisms is maintained. However, there are times when the physiological, biochemical, and/or environmental conditions permit the microorganisms to tip that balance, thereby causing an infection.

Nails and surrounding tissues are susceptible to many types of microbial infection. For example, bacterial nail infections include paronychia, which may also be related to viral infections. Certain fungal infections known as tinea infections are caused by mold-like fungi that thrive in warm, moist areas, thriving on the dead tissues of hair, nails, and outer skin layers. Tinea infections include tinea unguum, onychomycosis, affects the fingernails or toenails. Tinea infections are contagious and can be passed through direct contact or by contact with clothing, from shower and pool surfaces, and even from pets. Reoccurrences of nail infections are often frequent. For some subjects, such as those also diagnosed with diabetes or circulatory problems, or obese subjects, tinea infections and their treatment can be quite serious. In general, there are at least 3 different types of nail infections caused by fungi. The most common infection is frequently caused by Trichophyton rubrum and affects the nail bed and the area beneath the nail. Another type of infection affects only the nail surface and creates white or light colored patches. This second type of fungal infection is unusual and represents only about 10% of the reported cases. A third type of fungal infection affects the nail root and usually afflicts persons with impaired immune defense. A fourth (and unusual) type is caused by an infection of yeast fungi. Infections by yeast most often only affect nails that already are infected or damaged in some way.

The fungi are invasive to the keratin nail tissue. Apart from becoming discolored and brittle, the nail may often separate from the nail bed. In addition, pain and difficulty in wearing foot apparel is often experienced. Initially, the disease affects only one nail, typically one nail of the foot, and is thereafter spread to more nails. The palms of the hands and the soles of the feet may frequently be affected as well. When the skin is affected, red spots frequently occur and the skin may peel off. Nail fungal infections are one of the hardest forms of external infection to treat, of which infections of toe nails are the most difficult to treat.

### SUMMARY

In one aspect, a combination treatment method for treating a bacterial or fungal nail infection includes providing one or more first composition components for mixing with diluent to formulate a first topical nail composition comprising a nail lacquer for application to an infected nail in a thin layer. The one or more first composition components may include mupirocin. The method may further include providing one or more second composition components for mixing with diluent to formulate a second topical nail composition comprising a nail lacquer for application to the infected nail in a layer. The one or more second composition components may include tetracycline, nitrofurantoin, or combination thereof.

In various embodiments, the one or more first composition components further include itraconazole and the one or more second composition components comprise tetracycline. In one embodiment, the one or more first composition components may further include doxycycline and clotrimazole and the one or more second composition components include nitrofurantoin.

In one embodiment, the method may further include providing a third topical nail composition for application to the infected nail in a thin layer prior to application of the first topical nail composition and/or the second topical nail composition. The third topical nail composition may include a solution including a NSAID and DMSO. The NSAID may comprise diclofenac. In one example, the third topical nail composition includes approximately 0.05% to approximately 5% diclofenac by weight and approximately 25% to approximately 50% DMSO by weight.

In one embodiment, the method includes formulating the first topical nail composition comprising mixing diluent with the one or more first composition components and/or formulating the second topical nail composition comprising mixing diluent with the one or more second composition components. In one example, the one or more first composition components further comprise itraconazole and formulating the first topical nail composition comprises mixing approximately 10 mg to approximately 50 mg mupirocin and approximately 20 mg to approximately 100 mg itraconazole per 15 ml diluent. Formulating the second topical nail composition may include mixing approximately 500 mg to approximately 1000 mg tetracycline per 15 ml diluent. In one example, the one or more first composition components further comprise doxycycline and clotrimazole and formulating the first topical nail composition comprises mixing approximately 50 mg to approximately 200 mg doxycycline, approximately 10 mg to approximately 50 mg mupirocin, and approximately 15 mg to approximately 50 mg clotrimazole per 15 ml diluent. Formulating the second topical nail composition may include mixing approximately 10 mg to approximately 100 mg nitrofurantoin per 15 ml of diluent. In one example, the method further includes applying the first topical nail composition to infected nails in a first daily administration and the second topical nail composition to infected nails in a separate, second daily administration.

In another aspect, a combination treatment method for treating a bacterial or fungal nail infection includes providing first composition components for mixing with diluent to formulate a first topical nail composition comprising a nail lacquer for application to an infected nail in a thin layer and second compositions components for mixing with diluent to formulate a second topical nail composition comprising a nail lacquer for application to the infected nail in a thin layer. The first composition components may include mupirocin, itraconazole, urea, and tobramycin, colistimethate, gentamycin, or combination thereof. The second composition components include urea and one or more of voriconazole or itraconazole.

In various embodiments, the method may further include providing a third topical nail composition for application to the infected nail in a thin layer prior to application of the first topical nail composition and/or the second topical nail composition. The third topical nail composition may include a solution including a NSAID and DMSO. In one example, the NSAID comprises diclofenac. In a further example, the third topical nail composition comprises approximately 0.05% to approximately 5% diclofenac by weight and approximately 25% to approximately 50% DMSO by weight.

In one embodiment, the method further includes formulating the first topical nail composition comprising mixing diluent with the first composition components and/or formulating the second topical nail composition comprising mixing diluent with the second composition components. In one example, formulating the first topical nail composition comprises mixing approximately 0.6 g to approximately 1.8 g tobramycin, approximately 10 mg to approximately 50 mg mupirocin, approximately 20 mg to approximately 100 mg itraconazole, and approximately 100 mg to approximately 1000 mg urea per 15 ml diluent, and formulating the second topical nail composition comprises mixing approximately 100 mg to approximately 1000 mg urea and at least one of approximately 100 mg to approximately 300 mg voriconazole or approximately 50 mg to approximately 200 mg itraconazole per 15 ml diluent. In another example, formulating the first topical nail composition comprises mixing approximately 40 mg to approximately 120 mg gentamicin, approximately 10 mg to approximately 50 mg mupirocin, approximately 20 mg to approximately 100 mg itraconazole, and approximately 100 mg to approximately 1000 mg urea per 15 ml diluent, and formulating the second topical nail composition comprises mixing approximately 100 mg to approximately 1000 mg urea and at least one of approximately 100 mg to approximately 300 mg voriconazole or approximately 50 mg to approximately 200 mg itraconazole per 15 ml diluent. In another example, formulating the first topical nail composition comprises mixing approximately 100 mg to approximately 400 mg colistimethate, approximately 10 mg to approximately 50 mg mupirocin, approximately 20 mg to approximately 100 mg itraconazole, and approximately 100 mg to approximately 1000 mg urea per 15 ml diluent, and formulating the second topical nail composition comprises mixing approximately 100 mg to approximately 1000 mg urea and at least one of approximately 100 mg to approximately 300 mg voriconazole or approximately 50 mg to approximately 200 mg itraconazole per 15 ml diluent. In one example, the method further comprises applying the first topical nail composition to the infected nail in a first daily administration and the second topical nail composition to the infected nail in a separate, second daily administration.

In still another aspect, a combination treatment method for treating a bacterial or fungal nail infection includes providing first composition components and one or more second composition components for mixing with diluent to formulate a first topical composition and a second topical composition, each comprising a solution for administration to an infected nail in a footbath or via irrigation. The first composition components may include doxycycline, mupirocin, and clotrimazole. The second the one or more second composition components may include one or both of clindamycin or colistimethate.

In one embodiment, the method may further include providing a topical nail composition for application to the infected nail in a thin layer prior to application of the first topical composition and/or prior to the application of the second topical composition. The topical nail composition may include a solution including a NSAID and DMSO. In one example, the topical nail composition comprises approximately 0.05% to approximately 5% by weight of the NSAID and approximately 25% to approximately 50% DMSO by weight.

In various embodiments, the method further includes formulating the first topical composition comprising mixing diluent with the first composition components and/or formulating the second topical composition comprising mixing diluent with the second composition components. In one example, formulating the first topical composition includes mixing approximately 50 mg to approximately 150 mg doxycycline, approximately 15 mg to approximately 40 mg mupirocin, and approximately 25 mg to approximately 75 mg clotrimazole, and approximately 0.5 L to approximately 3 L diluent, and formulating the second topical composition comprises mixing approximately 400 mg to approximately 700 mg clindamycin, and approximately 0.5 L to approximately 3 L diluent. In one example, formulating the first topical composition comprises mixing approximately 400 mg to approximately 1200 mg urea with the first composition components and diluent and formulating the second topical composition comprises mixing the approximately 400 mg to approximately 1200 mg urea with the second composition components and diluent. In a further example, the method further includes providing a topical nail composition for application to the infected nail in a thin layer prior to application of the first topical composition and/or prior to the application of the second topical composition. The topical nail composition may include a solution including a NSAID and DMSO. In one such example, the topical nail composition comprises approximately 0.05% to approximately 5% by weight of the NSAID and approximately 25% to approximately 50% DMSO by weight. In one example the method may include administering the first topical composition in a first daily administration, administering the second topical composition in a separate, second daily administration, and applying the topical nail composition to the infected nail in a thin layer prior to administration of the first composition, second composition, or both. In one example, formulating the first topical composition comprises mixing approximately 50 mg to approximately 150 mg doxycycline, approximately 15 mg to approximately 40 mg mupirocin, approximately 25 mg to approximately 75 mg clotrimazole and approximately 0.5 L to approximately 3 L diluent, and formulating the second topical composition comprises mixing approximately 200 mg to approximately 500 mg colistimethate and approximately 0.5 L to approximately 3 L diluent.

In various embodiments, the one or more second composition components comprise clindamycin. The method may further include providing one or more third composition components for mixing with diluent to formulate a third composition comprising a solution for administration to a nail in a footbath or by irrigation. The one or more third composition components may include colistimethate. In one example, the method may further include formulating the first topical composition including mixing approximately 50 mg to approximately 150 mg doxycycline, approximately 15 mg to approximately 40 mg mupirocin, approximately 25 mg to approximately 75 mg clotrimazole, and approximately 0.5 L to approximately 3 L diluent; formulating the second topical composition including mixing approximately 400 mg to approximately 700 mg clindamycin, and approximately 0.5 L to approximately 3 L diluent; and formulating the third topical composition including mixing approximately 200 mg to approximately 500 mg colistimethate, and approximately 0.5 L to approximately 3 L diluent. In one example, formulating one or more of the first topical composition, second topical composition, or third topical composition further includes mixing approximately 400 mg to approximately 1200 mg urea with the diluent and respective composition components. In another example, the method may include providing a topical nail composition for application to the infected nail in a thin layer prior to application of the first topical composition and/or prior to the application of the second topical composition, wherein the topical nail composition comprising a solution including a NSAID and DMSO. In one example, the topical nail composition comprises approximately 0.05% to approximately 5% by weight of the NSAID and approximately 25% to approximately 50% DMSO by weight. In a further example, the method includes administering the first topical composition in a first daily administration, administering the second topical composition in a separate, second daily administration, administering the third topical composition in a separate, third daily administration, and administering the topical nail composition to the infected nail in a thin layer prior to one or more of the first, second, or third daily administrations.

In yet another aspect, a combination treatment method for treating a bacterial or fungal nail infection includes providing one or more first composition components and one or more second composition components for mixing with diluent to formulate a first topical composition and a second topical composition, each comprising a solution for administration to an infected nail in a footbath or via irrigation. The one or more first composition components may include mupirocin and the one or more second composition components may include tetracycline, nitrofurantoin, or combination thereof.

In one example, the one or more first composition components further comprise itraconazole and the one or more second composition components comprise tetracycline. In one example, the one or more first composition components further comprise doxycycline and clotrimazole and the one or more second composition components comprise nitrofurantoin.

In various embodiments, the method may further include providing a topical nail composition for application to the infected nail in a thin layer prior to administration of the first topical composition and/or the second topical composition, the topical nail composition comprising a solution including a NSAID and DMSO. In one example, the NSAID comprises diclofenac. In another example, the topical nail composition comprises approximately 0.05% to approximately 5% diclofenac by weight and approximately 25% to approximately 50% DMSO by weight.

In one embodiment, the method may further include formulating the first topical composition and/or formulating the second topical composition. In one example, the one or more first composition components further include itraconazole and formulating the first topical composition comprises mixing approximately 10 mg to approximately 50 mg mupirocin, approximately 20 mg to approximately 100 mg itraconazole, and approximately 0.5 L to approximately 3 L diluent, and formulating the second topical composition comprises mixing approximately 500 mg to approximately 1000 mg tetracycline and approximately 0.5 L to approximately 3 L diluent. In one embodiment, the one or more first composition components further include doxycycline and clotrimazole, and formulating the first topical composition comprises mixing approximately 50 mg to approximately 200 mg doxycycline, approximately 10 mg to approximately 50 mg mupirocin, approximately 15 mg to approximately 50 mg clotrimazole, and approximately 0.5 L to approximately 3 L diluent, and formulating the second topical composition comprises mixing approximately 10 mg to approximately 100 mg nitrofurantoin and approximately 0.5 L to approximately 3 L diluent. In one embodiment, the method further includes administering the first topical composition to infected nails in a first daily administration and administering the second topical composition to infected nails in a separate, second daily administration.

In still yet another aspect, a method of treating a fungal or bacterial nail infection in a footbath or via irrigation includes formulating a footbath solution comprising mixing mupirocin, doxycycline, and diluent, and administering the footbath solution to an infected nail by submerging the infected nail in the footbath solution or by irrigation of the infected nail with the footbath solution.

In various embodiments, formulating the footbath solution comprises mixing approximately 150 mg to approximately 250 mg doxycycline and approximately 25 mg to approximately 75 mg mupirocin. In one example, formulating the footbath solution further comprises mixing approximately 100 mg to approximately 300 mg voriconazole. In one example, formulating the footbath solution further comprises mixing approximately 400 mg to approximately 700 mg clindamycin. In one example, formulating the footbath solution further comprises mixing approximately 200 mg to approximately 500 mg colistimethate. In another example, formulating the footbath solution further comprises mixing approximately 30 to approximately 75 mg clotrimazole. In one example, formulating the footbath solution comprises mixing approximately 50 mg to approximately 150 mg doxycycline, approximately 15 mg to approximately 40 mg mupirocin, approximately 15 mg to approximately 40 mg clotrimazole, and approximately 400 mg to approximately 700 mg clindamycin.

### DESCRIPTION

The present disclosure describes topical nail compositions and topical compositions for bath or irrigation for topical application to nails to treat bacterial or fungal nail infections. In some embodiments, the compositions may also be used to treat foot infections in addition to nail infections. The present disclosure also describes combination treatments including one or more first compositions and one or more second compositions wherein the one or more first and/or second compositions comprise one or more topical nail compositions and/or one or more topical compositions.

### Topical Nail Compositions

The topical nail compositions may be referred to as nail lacquers for direct application to the nail tissue. The nail lacquers may include one or more anti-infective agents formulated for topical application to nail tissue. In some embodiments, a nail lacquer may include additives such as thickening agents, plasticizers, polymers, volatile organic compounds, or other additives to promote effective localization of the medication following application. In some embodiments, a nail lacquer may comprise a solution, which may be a suspension or mixture. In some embodiments, the nail lacquer may lack traditional lacquer additives. In various embodiments, nail lacquers may comprise an aqueous solution formulated to be applied to a nail surface whereon the aqueous carrier evaporates or is absorbed. The solution may have a fluid or semi-fluid consistency. In some embodiments, the nail lacquer may be thickened with a viscosity agent to increase viscosity for application. In some embodiments, the nail lacquer may comprise a solution comprising a cream, lotion, gel, or ointment.

In various embodiments, the topical nail composition, which may also be referred to as a nail lacquer for topical application directly to nails, to treat of a bacterial or fungal nail infection comprises one or more anti-infective agents and a diluent.

The anti-infective agent may include one or more antibacterial agents, antifungal agents, antiviral agents, such as pharmaceutical drug antibiotics, antifungals, and antivirals, or combinations thereof.

In various embodiments, the topical nail composition comprises an antibacterial agent, alone or in combination with one or more antifungal agents, comprising one or more enicillins, cephalosporins, fluoroquinolones, aminoglycosides, monobactams, carbapenems, macrolides, and other antibacterials. For example, the antibacterial agent may include one or more antibacterials selected from afenide, amikacin, amoxicillin, ampicillin, arsphenamine, azithromycin, azlocillin, aztreonam, bacampicillin, bacitracin, carbacephem (loracarbef), carbenicillin, cefaclor, cefadroxil, cefalotin, cefamandole, cefazolin, cefdinir, cefditoren, cefepime, cefixime, cefoperazone, cefotaxime, cefoxitin, cefpodoxime, cefprozil, ceftazidime, ceftibuten, ceftizoxime, ceftobiprole, ceftriaxone, cefuroxime, cephalexin, chloramphenicol, chlorhexidine, ciprofloxacin, clarithromycin, clavulanic acid, clindamycin, cloxacillin, colimycin, colistimethate teicoplanin, colistin, demeclocycline, dicloxacillin, dirithromycin, doripenem, doxycycline, efprozil, enoxacin, ertapenem, erythromycin, ethambutol, flucloxacillin, fosfomycin, furazolidone, gatifloxacin, geldanamycin, gentamicin, grepafloxacin, herbimycin, imipenem, isoniazid, kanamycin, levofloxacin, lincomycin, linezolid, lomefloxacin, meropenem, meticillin, meticillin, mezlocillin, minocycline, mitomycin, moxifloxacin, mupirocin, nafcillin, neomycin, netilmicin, nitrofurantoin, norfloxacin, ofloxacin, oxacillin, oxytetracycline, paromomycin, penicillin G, penicillin V, piperacillin, pivmecillinam, platensimycin, polymyxin B, prontosil, pvampicillin, pyrazinamide, quinupristin/dalfopristin, rifampicin, rifampin, roxithromycin, sparfloxacin, spectinomycin, spiramycin, sulbactam, sulfacetamide, sulfamethizole, sulfamethoxazole, sulfanilimide, sulfisoxazole, sulphonamides, sultamicillin, telithromycin, tetracycline, thiamphenicol, ticarcillin, tobramycin, trimethoprim, trimethoprim-sulfamethoxazole, troleandomycin, trovafloxacin, or a combination thereof. In some embodiments, the antibacterial agent is selected from mupirocin, gentamycin, tobramycin, or combinations thereof. In one embodiment, the antibacterial agent includes an aminoglycoside.

In various embodiments, the topical nail composition may comprise between 0.01% and 20% by weight antibacterial agent, such as between approximately 0.01% and approximately 5%, between approximately 0.01% and approximately 3%, between approximately 0.01% and approximately 1%, between approximately 0.01% and approximately 0.25%, between approximately 0.01% and approximately 0.15%, or between approximately 0.05% and approximately 0.15%, between 0.1% and 10%, between approximately 0.1% and approximately 0.5%, between approximately 0.1% and approximately 0.2%, between approximately 0.2% and approximately 0.8%, between approximately 0.2% and approximately 0.6%, between approximately 0.2% and approximately 0.4%, between approximately 0.3% and approximately 1%, between approximately 0.3% and approximately 0.8%, between approximately 0.3% and approximately 0.6%, between approximately 0.4% and approximately 1%, between approximately 0.5% and approximately 1%, between approximately 0.5% and approximately 8%, between approximately 0.6% and approximately 1%, between approximately 0.6% and approximately 0.8%, between approximately 0.8% and approximately 1%, between approximately 1% and approximately 10%, between approximately 1% and approximately 8%, between approximately 1% and approximately 5%, between approximately 1% and approximately 3%, between approximately 3% and approximately 10%, between approximately 3% and approximately 8%, or between approximately 3% and approximately 5%, between 5% and 10%, between approximately 5% and approximately 8%, between approximately 6% and approximately 10%, between approximately 6% and approximately 8%, between approximately 7% and approximately 10%, between approximately 8% and approximately 10%, between approximately 10% and approximately 20%, between approximately 10% and approximately 15%, between approximately 10% and approximately 12%, between approximately 12% and approximately 15%, or between approximately 15% and approximately 20% of the topical nail composition by weight. In some embodiments, the amount of antibacterial by weight may be approximately 0.01%, approximately 0.05%, approximately 0.1%, approximately 0.5%, approximately 1%, approximately 1.5%, approximately 2%, approximately 2.5%, approximately 3%, approximately 3.5%, approximately 4%, approximately 4.5%, approximately 5%, approximately 5.5%, approximately 6%, approximately 6.5%, approximately 7%, approximately 7.5%, approximately 8%, approximately 8.5%, approximately 9%, approximately 9.5%, approximately 10%, approximately 11%, approximately 12%, approximately 13%, approximately 14%, approximately 15%, approximately 17%, approximately 19%, approximately 20%, or any other percentage between approximately 0.01% and 20% by weight of the topical nail composition. In various embodiments, a topical nail composition dosage may comprise up to 2500 mg antibacterial. For example, antibacterial agent may be present in an amount between approximately 10 mg and approximately 2500 mg, such as between approximately 10 and approximately 2000 mg, between approximately 20 mg and approximately 100 mg, between approximately 50 mg and approximately 120, between approximately 80 mg and approximately 120 mg, between approximately 100 mg and approximately 150 mg, or between approximately 120 mg and approximately 170 mg, between approximately 150 mg and approximately 200 mg, between approximately 180 mg and approximately 220 mg, between approximately 200 mg and approximately 250 mg, between approximately 250 mg and approximately 280 mg, between approximately 280 mg and approximately 300 mg, between approximately 300 mg and approximately 360 mg, between approximately 360 mg and approximately 440 mg, between approximately 440 mg and approximately 500 mg, between approximately 500 mg and approximately 600 mg, between approximately 600 mg and approximately 800 mg, between approximately 800 mg and approximately 1000 mg, between approximately 1000 mg and approximately 1200 mg, between approximately 1200 mg and approximately 1400 mg, between approximately 1400 mg and approximately 1600 mg, between approximately 1600 mg and approximately 1800 mg, between approximately 1800 mg and approximately 2000 mg, between approximately 2000 mg and approximately 2500 mg, between approximately 100 mg and approximately 500 mg, between approximately 250 mg and approximately 750 mg, between approximately 500 mg and approximately 1000 mg, between approximately 1000 mg and approximately 1500 mg, or between approximately 750 mg and approximately 1500 mg per approximately 10 ml to approximately 20 ml diluent.

In various embodiments, the one or more anti-infective agents comprises an antibacterial agent selected from one or more antibacterials comprising Vancomycin, ciprofloxacin, levofloxacin, azithromycin, clindamycin, doxycycline, mupirocin, ceftriaxone, colistimethate, tobramycin, cefepime, gentamicin, streptomycin, sulfamethoxazole/trimethoprim, tetracycline, nitrofurantoin.

The topical nail composition may include commercially available vancomycin, such as Vancomycin Hydrochloride for Injection, USP, which is a lyophilized powder for preparing intravenous (IV) infusions. The powder may be provided in vials (e.g., bottles) containing the equivalent of 500 mg, 1 g, 5 grams, 10 grams vancomycin base. Other formats may be used, for example Vancomycin Hydrochloride USP powder for oral solution, equivalent to 3.75 g, 7.5 g or 15 g vancomycin, and diluent, which may be a flavored, e.g., grape-flavored, diluent for reconstitution; Vancomycin Intravenous Solution, e.g., vancomycin hydrochloride 5 mg/mL, sodium chloride 9 mg/mL; or Vancomycin Capsules; or bulk powder. In various embodiments, vancomycin may be present in an amount between approximately 1 mg and approximately 100 mg, such as between approximately 5 mg and approximately 50 mg or between approximately 15 mg and approximately 40 mg per approximately 10 ml to approximately 20 ml diluent.

The topical nail composition may include commercially available ciprofloxacin, such Ciprofloxacin Hydrochloride Solution/Drops; Ciprofloxacin Hydrochloride Tablets; Ciprofloxacin Tablets, e.g., 500 mg or 100 mg; Ciprofloxacin Hydrochloride Suspension; Ciprofloxacin Injection, USP, e.g., Ciprofloxacin Injection, USP, 20 mL, 200 mg, 1% and 40 mL or 400 mg, 1%, for intravenous injection and infusion, Premix 100 mL in 5% Dextrose, 200 mg, 0.2% and 200 mL in 5% Dextrose or 400 mg, 0.2%, for intravenous infusion; or bulk powder. In various embodiments, ciprofloxacin may be present in an amount between approximately 10 mg and approximately 1500 mg, such as between approximately 200 mg and approximately 1000 mg, approximately 100 mg, approximately 200 mg, approximately 300 mg, approximately 400 mg, or approximately 500 mg per approximately 10 ml to approximately 20 ml diluent.

The topical nail composition may include commercially available levofloxacin, such as Levofloxacin Injection, which may be supplied in single-use vials containing a concentrated solution with the equivalent of 500 mg of levofloxacin USP in 20 mL vials and 750 mg of levofloxacin USP in 30 mL vials; Levofloxacin Solution/Drops; Levofloxacin Tablet; or bulk powder. In various embodiments, levofloxacin may be present in an amount between approximately 10 mg and approximately 1500 mg, such as between approximately 200 mg and approximately 1000 mg or between approximately 400 mg and approximately 750 mg, approximately 250 mg, approximately 500 mg, or approximately 750 mg per approximately 10 ml to approximately 20 ml diluent.

The topical nail composition may include commercially available azithromycin, such as Azithromycin for Injection USP, which may be supplied in lyophilized form under a vacuum in a 10 mL vial equivalent to 500 mg of azithromycin for intravenous administration including sodium hydroxide and 413.6 mg citric acid; Azithromycin for Oral Suspension, USP, which may be supplied for suspension in 100 mg/5 mL or 200 mg/5 mL; Azithromycin Tablets; or bulk powder. In various embodiments, Azithromycin may be present in an amount between approximately 10 mg and approximately 1500 mg, such as between approximately 200 mg and approximately 1000 mg, approximately 100 mg, approximately 200 mg, approximately 300 mg, approximately 400 mg, or approximately 500 mg per approximately 10 ml to approximately 20 ml diluent.

The topical nail composition may include commercially available clindamycin, such as Clindamycin Phosphate Cream; Clindamycin Phosphate Gel; Clindamycin Phosphate Suspension; Clindamycin Phosphate Injection Solution; Clindamycin Phosphate for Injection; or bulk powder. In various embodiments, clindamycin may be present in an amount between approximately 10 mg and approximately 1500 mg, such as between approximately 200 mg and approximately 1000 mg or between approximately 400 mg and approximately 700 mg, approximately 100 mg, approximately 200 mg, approximately 300 mg, approximately 400 mg, approximately 500 mg, approximately 600 mg, or approximately 700 mg per approximately 10 ml to approximately 20 ml diluent. The clindamycin may include a bulk powder, colistimethate for injection, solution, ground tablets, or a commercially available ointment or cream, for example. Clindamycin may comprise clindamycin phosphate topical solution, USP 1%.

The topical nail composition may include commercially available doxycycline, such as Doxycycline Hyclate tablets; Doxycycline Hyclate Tablets; Doxycycline Hyclate Pellets; Doxycycline for Suspension; Doxycycline Hyclate Powder for Suspension; or bulk powder. In various embodiments, doxycycline may be present in an amount between approximately 10 mg and approximately 500 mg, such as between approximately 25 mg and approximately 250 mg or between approximately 50 mg and approximately 150 mg, approximately 50 mg, approximately, 100 mg, approximately 150 mg, or approximately 200 mg per approximately 10 ml to approximately 20 ml diluent.

The topical nail composition may include commercially available mupirocin, such as Mupirocin Ointment; Mupirocin Cream; or bulk powder. In various embodiments, mupirocin may be present in an amount between approximately 1 mg and approximately 120 mg, such as between approximately 5 mg and approximately 100 mg or between approximately 30 mg and approximately 75 mg, approximately 20 mg, approximately 30 mg, approximately 40 mg, or approximately 50 mg per approximately 10 ml to approximately 20 ml diluent.

The topical nail composition may include commercially available cefepime, such as Cefepime Hydrochloride Injection, Powder, for Solution, supplied in 500 mg, 1 g, and 2 g vials; Cefepime Hydrochloride Injection Solution; or bulk powder. In various embodiments, cefepime may be present in an amount between approximately 500 mg and approximately 2500 mg, such as between approximately 1000 mg and approximately 2000 mg or between approximately 500 mg and approximately 1000 mg per approximately 10 ml to approximately 20 ml diluent.

The topical nail composition may include commercially available streptomycin, such as Streptomycin for Injection USP, which may be supplied in 1 g vials; Streptomycin Injection, Powder, Lyophilized, for Solution; or bulk powder. In various embodiments, streptomycin may be present in an amount between approximately 500 mg and approximately 2500 mg, such as between approximately 1000 mg and approximately 2000 mg or between approximately 500 mg and approximately 1000 mg per approximately 10 ml to approximately 20 ml diluent.

The topical nail composition may include commercially available sulfamethoxazole /trimethoprim, such as Sulfamethoxazole and Trimethoprim Tablets; Sulfamethoxazole and Trimethoprim Injection; Sulfamethoxazole and Trimethoprim Suspension; or bulk powder.

The topical nail composition may include tetracycline, which may include a commercially available tetracycline format or bulk powder. In various embodiments, tetracycline may be present in an amount between approximately 500 mg and approximately 2500 mg, such as between approximately 1000 mg and approximately 2000 mg or between approximately 1250 mg and approximately 1750 mg, approximately 500 mg, approximately 1000 mg, or approximately 1500 mg per approximately 10 ml to approximately 20 ml diluent. In various embodiments, the tetracycline comprises tetracycline bulk powder, injection solution, cream, ointment, or ground tablets.

The topical nail composition may include nitrofurantoin, which may include a commercially available nitrofurantoin format or bulk powder. In various embodiments, nitrofurantoin may be present in an amount between approximately 10 mg and approximately 250 mg, such as between approximately 25 mg and approximately 150 mg or between approximately 50 mg and approximately 100 mg per approximately 10 ml to approximately 20 ml diluent. The nitrofurantoin may include a bulk powder, solution, ground tablets, or a commercially available nitrofurantoin ointment or cream, for example.

In various embodiments, the topical nail composition comprises an antifungal agent, alone or in combination with one or more antibacterial agents, wherein the an antifungal agent includes one or more antifungals selected from one or more categories of antifungal agents including azoles (imidazoles), antimetabolites, allylamines, morpholine, glucan synthesis inhibitors (echinocandins), polyenes, benoxaaborale; other antifungal/onychomycosis agents, and new classes of antifungal/onychomycosis agents. For example, the anti-fungal agent may comprise one or more antifungals selected from abafungin, albaconazole, amorolfin, amphotericin b, anidulafungin, bifonazole, butenafine, butoconazole, candicidin, caspofungin, ciclopirox, clotrimazole, econazole, fenticonazole, filipin, fluconazole, flucytosine, griseofulvin, haloprogin, hamycin, isavuconazole, isoconazole, itraconazole, ketoconazole, micafungin, miconazole, naftifine, natamycin, nystatin, omoconazole, oxiconazole, polygodial, posaconazole, ravuconazole, rimocidin, sertaconazole, sulconazole, terbinafine, terconazole, tioconazole, tolnaftate, undecylenic acid, voriconazole, or a combination thereof. In some embodiments, the antibacterial agent is selected from one or more azoles. In one embodiment, the antifungal agent is selected from itraconazole, voriconazole, or combination thereof.

In various embodiments, the topical nail composition may comprise between approximately 0.01% and approximately 20% by weight antifungal agent, such as between approximately 0.01% and approximately 5%, between approximately 0.01% and approximately 3%, between approximately 0.01% and approximately 1%, between approximately 0.01% and approximately 0.25%, between approximately 0.01% and approximately 0.15%, or between approximately 0.05% and approximately 0.15%, between 0.1% and 10% by weight, between approximately 0.1% and approximately 0.5%, between approximately 0.1% and approximately 0.2%, between approximately 0.2% and approximately 0.8%, between approximately 0.2% and approximately 0.6%, between approximately 0.2% and approximately 0.4%, between approximately 0.3% and approximately 1%, between approximately 0.3% and approximately 0.8%, between approximately 0.3% and approximately 0.6%, between approximately 0.4% and approximately 1%, between approximately 0.5% and approximately 1%, between approximately 0.5% and approximately 8%, between approximately 0.6% and approximately 1%, between approximately 0.6% and approximately 0.8%, between approximately 0.8% and approximately 1%, between approximately 1% and approximately 3%, between approximately 1% and approximately 10%, between approximately 1% and approximately 8%, between approximately 1% and approximately 5%, between approximately 1% and approximately 3%, between approximately 3% and approximately 10%, between approximately 3% and approximately 8%, or between approximately 3% and approximately 5%, between 5% and 10%, between approximately 5% and approximately 8%, between approximately 6% and approximately 10%, between approximately 6% and approximately 8%, between approximately 7% and approximately 10%, between approximately 8% and approximately 10%, between approximately 10% and approximately 20%, between approximately 10% and approximately 15%, between approximately 10% and approximately 12%, between approximately 12% and approximately 15%, or between approximately 15% and approximately 20% of the topical nail composition by weight. In some embodiments, the amount of antifungal by weight may be approximately 0.01%, approximately 0.05%, approximately 0.1%, approximately 0.5%, approximately 1%, approximately 1.5%, approximately 2%, approximately 2.5%, approximately 3%, approximately 3.5%, approximately 4%, approximately 4.5%, approximately 5%, approximately 5.5%, approximately 6%, approximately 6.5%, approximately 7%, approximately 7.5%, approximately 8%, approximately 8.5%, approximately 9%, approximately 9.5%, approximately 10%, approximately 11%, approximately 12%, approximately 13%, approximately 14%, approximately 15%, approximately 17%, approximately 19%, approximately 20%, or any other percentage between approximately 0.01% and 20% by weight of the topical nail composition. In various embodiments, a topical nail composition dosage may include up to 2500 mg antifungal agent, which may include one or more antifungals. For example, antifungal agent may be present in an amount between approximately 10 mg and approximately 2500 mg, such as between approximately 10 and approximately 2000 mg, between approximately 20 mg and approximately 100 mg, between approximately 50 mg and approximately 120, between approximately 80 mg and approximately 120 mg, between approximately 100 mg and approximately 150 mg, or between approximately 120 mg and approximately 170 mg, between approximately 150 mg and approximately 200 mg, between approximately 180 mg and approximately 220 mg, between approximately 200 mg and approximately 250 mg, between approximately 250 mg and approximately 280 mg, between approximately 280 mg and approximately 300 mg, between approximately 300 mg and approximately 360 mg, between approximately 360 mg and approximately 440 mg, between approximately 440 mg and approximately 500 mg, between approximately 500 mg and approximately 600 mg, between approximately 600 mg and approximately 800 mg, between approximately 800 mg and approximately 1000 mg, between approximately 1000 mg and approximately 1200 mg, between approximately 1200 mg and approximately 1400 mg, between approximately 1400 mg and approximately 1600 mg, between approximately 1600 mg and approximately 1800 mg, between approximately 1800 mg and approximately 2000 mg, between approximately 2000 mg and approximately 2500 mg, between approximately 100 mg and approximately 500 mg, between approximately 250 mg and approximately 750 mg, between approximately 500 mg and approximately 1000 mg, between approximately 1000 mg and approximately 1500 mg, or between approximately 750 mg and approximately 1500 mg per approximately 10 ml to approximately 20 ml diluent.

In various embodiments, the one or more anti-infective agents comprises an antifungal agent selected from one or more antifungals comprising fluconazole, itraconazole, voriconazole, amphotericin, nystatin, clotrimazole, econazole, or ketoconazole.

The topical nail composition may include commercially available fluconazole, such as Fluconazole in Dextrose Inject, Solution; Fluconazole in Sodium Chloride Injection, Solution; Fluconazole Injection; Fluconazole Powder, for Suspension; Fluconazole Tablets; or bulk powder. In various embodiments, fluconazole may be present in an amount between approximately 10 mg and approximately 250 mg, such as between approximately 25 mg and approximately 150 mg or between approximately 50 mg and approximately 100 mg, approximately 50 mg, approximately 100 mg, approximately 150 mg, approximately 200 mg per approximately 10 ml to approximately 20 ml diluent.

The topical nail composition may include commercially available itraconazole, such as Itraconazole Capsule; Itraconazole Injection Solution; or bulk powder. In various embodiments, itraconazole may be present in an amount between approximately 5 mg and approximately 150 mg, such as between approximately 20 mg and approximately 150 mg or between approximately 25 mg and approximately 75 mg, approximately 50 mg, or approximately 100 mg per approximately 10 ml to approximately 20 ml diluent.

The topical nail composition may include commercially available voriconazole, such as Voriconazole Injection, Powder, Lyophilized, for Solution; Voriconazole Injection, Powder, for Solution; Voriconazole Powder; Voriconazole Tablet; or bulk powder. In various embodiments, voriconazole may be present in an amount between approximately 10 mg and approximately 750 mg, such as between approximately 50 mg and approximately 500 mg or between approximately 100 mg and approximately 300 mg, approximately 100 mg, approximately 150 mg, approximately 200 mg, or approximately 250 mg per approximately 10 ml to approximately 20 ml diluent.

The topical nail composition may include commercially available amphotericin, such as Amphotericin B injection, Lipid Complex; Amphotericin B Injection, Powder, Lyophilized, for Solution; or bulk powder. In various embodiments, amphotericin may be present in an amount between approximately 1 mg and approximately 120 mg, such as between approximately 5 mg and approximately 100 mg or between approximately 30 mg and approximately 75 mg, approximately 20 mg, approximately 30 mg, approximately 40 mg, or approximately 50 mg per approximately 10 ml to approximately 20 ml diluent.

The topical nail composition may include commercially available nystatin, such as Nystatin Cream; Nystatin Ointment; Nystatin Powder (Topical); or bulk powder.

The topical nail composition may include commercially available clotrimazole, such as Clotrimazole Cream; Clotrimazole Lotion; Clotrimazole Liquid; Clotrimazole Solution; or bulk powder. In various embodiments, clotrimazole may be present in an amount between approximately 5 mg and approximately 150 mg, such as between approximately 20 mg and approximately 150 mg or between approximately 25 mg and approximately 75 mg, approximately 20 mg, approximately 30 mg, approximately 40 mg, or approximately 50 mg per approximately 10 ml to approximately 20 ml diluent.

The topical nail composition may include commercially available econazole, such as Econazole Nitrate Cream; Econazole Nitrate Foam; or bulk powder.

The topical nail composition may include commercially available ketoconazole, such as Ketoconazole Foam; Ketoconazole Cream; Ketoconazole Suspension; Ketoconazole Tablet; Ketoconazole Suspension Shampoo; or bulk powder. In various embodiments, ketoconazole may be present in an amount between approximately 10 mg and approximately 750 mg, such as between approximately 50 mg and approximately 500 mg or between approximately 100 mg and approximately 300 mg, approximately 100 mg, approximately 150 mg, approximately 200 mg, or approximately 250 mg per approximately 10 ml to approximately 20 ml diluent.

As introduced above, in various embodiments, the antibacterial agent or one or more antibacterials thereof, the antifungal agent or one or more antifungals thereof, or combination thereof comprises a commercially available antibacterial or antifungal for injection.

In one example, the topical nail composition comprises a commercially available tobramycin as tobramycin for injection. Tobramycin for injection is commercially available in various strengths and volumes. For example, tobramycin for injection is currently available in as Tobramycin Injection, USP, 40 mg/1 mL (30 mL), 1.2 g multi-dose vials, 80 mg/2 mL vials, 240 mg/6 mL, 20 mg or 80 mg in 20 mg/2 mL vials. Tobramycin for injection may be provided as powder or solution and may include tobramycin sulfate. In various embodiments, tobramycin may be present in an amount between approximately 5 mg and approximately 300 mg, such as between approximately 20 mg and approximately 250 mg, approximately 25 mg and approximately 200 mg, approximately 50 mg and 100 mg, approximately 20 mg, approximately 40 mg, approximately 60 mg, approximately 80 mg, approximately 100 mg, approximately 120 mg, approximately 160 mg, or approximately 220 mg per approximately 10 ml to approximately 20 ml diluent.

In any of the above or another example, the topical nail composition comprises a commercially available voriconazole as voriconazole for injection. Voriconazole for injection is commercially available in various strengths and volumes. For example, voriconazole for injection is currently available as Voriconazole for Injection supplied in a single dose vials as a sterile lyophilized powder equivalent to 200 mg voriconazole and 3200 mg sulfobutyl ether beta-cyclodextrin sodium (SBECD) and single doses vial as a sterile lyophilized white to off white cake or powder equivalent to 200 mg voriconazole and 3200 mg hydroxypropyl β-cyclodextrin (HPβCD). In various embodiments, voriconazole may be present in an amount between approximately 5 mg and approximately 400 mg, such as between approximately 20 mg and approximately 350 mg, approximately 50 mg and approximately 250 mg, approximately 100 mg and 200 mg, approximately 20 mg, approximately 50 mg, approximately 75 mg, approximately 100 mg, approximately 150 mg, approximately 180 mg, approximately 200 mg, or approximately 220 mg per approximately 10 ml to approximately 20 ml diluent.

In any of the above or another example, the topical nail composition comprises commercially available colistimethate as colistimethate for injection. Colistimethate for injection is commercially available in various strengths and volumes. For example, colistimethate for injection, USP is currently available in vials containing colistimethate sodium equivalent to 150 mg colistin base activity per vial, Colistimethate for Injection, USP, 150 mg. In various embodiments, colistimethate may be present in an amount between approximately 10 mg and approximately 1000 mg, such as between approximately 100 mg and approximately 750 mg or between approximately 200 mg and approximately 500 mg, approximately 200 mg, approximately 300 mg, approximately 400 mg, or approximately 500 mg per approximately 10 ml to approximately 20 ml diluent.

In any of the above or another example, the topical nail composition comprises commercially available ceftriaxone as ceftriaxone for injection. Ceftriaxone for injection is commercially available in various strengths and volumes. For example, Ceftriaxone for injection is currently available as Ceftriaxone for Injection, USP, 500 mg, 250 mg, 2 g, and 1 g. Ceftriaxone Injection, USP, Solution is also available premixed as a frozen, iso-osmotic, sterile, nonpyrogenic solution of ceftriaxone sodium in 1 g equivalent of ceftriaxone, iso-osmotic with approximately 1.9 g Dextrose Hydrous, USP, and 2 gm equivalent of ceftriaxone, iso-osmotic with approximately 1.2 g Dextrose Hydrous, USP. In various embodiments, ceftriaxone may be present in an amount between approximately 10 mg and approximately 1000 mg, such as between approximately 100 mg and approximately 750 mg or between approximately 200 mg and approximately 500 mg, approximately 200 mg, approximately 300 mg, approximately 400 mg, or approximately 500 mg per approximately 10 ml to approximately 20 ml diluent.

In any of the above or another example, the topical nail composition comprises commercially available levofloxacin as levofloxacin for injection. Levofloxacin for injection is commercially available in various strengths and volumes. For example, levofloxacin for injection is currently available in 500 mg/20 mL strength, 20 mL volume single use container, and in 250 mg/50 mL strength, 50 mL, 100 mL, and 150 mL single-use containers. In various embodiments, levofloxacin may be present in an amount between approximately 500 mg and approximately 2500 mg, such as between approximately 1000 mg and approximately 2000 mg or between approximately 500 mg and approximately 1000 mg, approximately 250 mg, approximately 500 mg, or approximately 750 mg per approximately 10 ml to approximately 20 ml diluent.

In any of the above or another example, the topical nail composition comprises commercially available linezolid as linezolid for injection. Linezolid for injection may be supplied as a ready-to-use sterile isotonic solution for intravenous infusion. For example, each container may contain 600 mg of linezolid in 300 mL of a clear, colorless to slightly yellow aqueous solution. Inactive ingredients may include: citric acid anhydrous USP 1.92 mg/mL, sodium chloride USP 9 mg/mL, sodium hydroxide NF 0.76 mg/mL, and water for injection USP. Sodium hydroxide NF and/or hydrochloric acid NF are typically used to adjust the pH. The sodium (Na+) content may be about 3.98 mg/mL (52 mEq/300-mL container). Zyvox for injection is supplied as a ready-to-use sterile isotonic solution for intravenous infusion. Each mL contains 2 mg of linezolid. Inactive ingredients are sodium citrate, citric acid, and dextrose in an aqueous vehicle for intravenous administration. The sodium (Na+) content is about 0.38 mg/mL (5 mEq per 300-mL bag; 3.3 mEq per 200-mL bag; and 1.7 mEq per 100-mL bag).

In these or other embodiments, the antibacterial agent or one or more antibacterials thereof, the antifungal agent or one or more antifungals thereof, or combination thereof comprises bulk powder, ground commercial oral tablets, a commercial oral, nasal, or injection solution or suspension, or commercially available topical cream, ointment, foam, lotion, or gel.

For example, the topical nail composition may include commercially available tobramycin in bulk powder, nasal solution, drops, or powder, ophthalmic suspension, solution, or drops, nebulizer formulas, or combination thereof. In this or another example, the topical nail composition may include voriconazole in a commercially available in bulk powder, oral suspension or powder for oral suspension, ground tablet, or combination thereof. In any of the above or another example, the topical nail composition may include linezolid in a commercially available bulk powder format, granules for oral suspension, oral solution, injection dosage, ground oral tablet, or combination thereof. In any of the above or another example, the topical nail composition may include mupirocin in a commercially available in cream, ointment, lotion, ground oral tablet, bulk powder, or combination thereof. In any of the above or another example, the topical nail composition may include itraconazole in a commercially available bulk powder, capsule, pellet, or combination thereof. In various embodiments, one or more of the anti-infective agents may be provided in a compounded tablet comprising bulk powder, ground oral tablets, or powder for suspension or solution.

In various embodiments, the topical nail composition comprises one or more anti-infective agents and one or more keratolitic agents selected form urea, salicylic acid, papain, or combinations thereof. For example, the topical nail composition may comprise one or more anti-infective agents and urea. In various embodiments, the topical nail composition may comprise between approximately 1% and approximately 30% by weight urea, such as between approximately 1% and approximately 20%, between approximately 1% and approximately 15%, between approximately 1% and approximately 10%, between approximately 1% and approximately 8%, between approximately 1% and approximately 7%, or between approximately 1% and approximately 6%, between 1% and 5% by weight, between approximately 1% and approximately 4%, between approximately 1% and approximately 3%, between approximately 1% and approximately 2%, between approximately 2% and approximately 4%, between approximately 3% and approximately 2%, between approximately 3% and approximately 4%, between approximately 3% and approximately 5%, or between approximately 3% and approximately 7%, between approximately 3% and approximately 10%, between approximately 4% and approximately 8%, between approximately 4% and approximately 10%, between approximately 6% and approximately 8%, between approximately 6% and approximately 10%, between approximately 8% and approximately 10%, between approximately 10% and approximately 20%, between approximately 10% and approximately 15%, between approximately 15% and approximately 20%, between approximately 20% and approximately 25%, between approximately 25% and approximately 30%, or between approximately 20% and approximately 30% of the topical nail composition by weight. In some embodiments, the amount of urea by weight may be approximately 1%, approximately 1.5%, approximately 1.8%, approximately 2%, approximately 2.1%, approximately 2.2%, approximately 2.3%, approximately 2.4%, approximately 2.5%, approximately 2.6%, approximately 2.7%, approximately 2.8%, approximately 2.9%, approximately 3%, approximately 3.1%, approximately 3.2%, approximately 3.3%, approximately 3.5%, approximately 3.7%, approximately 3.9%, approximately 4%, approximately 4.5%, approximately 5%, approximately 5.5%, approximately 6%, approximately 6.5%, approximately 7%, approximately 7.5%, approximately 8%, approximately 8.5%, approximately 9%, approximately 9.5%, approximately 10%, approximately 11%, approximately 12%, approximately 13%, approximately 14%, approximately 15%, approximately 17%, approximately 19%, approximately 20%, approximately 23%, approximately 25%, approximately 27%, approximately 29%, approximately 30% or any other percentage between approximately 1% and 30% by weight of the topical nail composition. In various embodiments, a topical nail composition may comprise urea in an amount between approximately 100 mg and approximately 1000 mg, such as between approximately 200 mg and approximately 800 mg or between approximately 400 mg and approximately 600 mg, or approximately 500 mg per approximately 10 ml to approximately 20 ml diluent.

As introduced above, the topical nail composition may include a diluent comprising one or more diluents. The diluent may be liquid, semi-liquid, or solid. For example, the diluent may include an aqueous, organic, or inorganic solution, cream, gel, ointment, lotion, emulsion, or paste. In some embodiments, the diluent includes a carrier or vehicle composition such as a base cream, ointment, gel, lotion, foam, solution, or suspension. The diluent may include lecithin, phospholipids, glycols, paraffin, fatty acids, carbopols, alcohols, lanolin, for example. In some embodiments, the diluent comprises an aqueous solution such as a saline solution. For example, the topical nail composition may comprise a diluent comprising sodium hydroxide solution, which may be a sterile solution, an alcohol, water, e.g., purified water, water for irrigation, water for injection, or a sterile water. In one embodiment, the diluent comprises a sodium chloride 0.09% solution (sterile). The diluent may be present in an amount sufficient to obtain the desired amount of active agents per unit weight or volume.

In various embodiments, the topical nail composition comprises Diluent dimethyl sulfoxide (DMSO). In some embodiments, a DMSO may be used as a diluent. A commercially available 45.5% DMSO solution, for example, may be used. Other percent concentrations of DMSO may be used, for example DMSO may be present in an amount between approximately 10% and approximately 50%, such as between approximately 15% and approximately 50%, between approximately 20% and approximately 50%, between approximately 25% and approximately 50%, between approximately 30% and approximately 50%, between approximately 35% and approximately 50%, between approximately 40% and approximately 50%, between approximately 20% and approximately 40%, or between approximately 20% and approximately 30%.

In various embodiments, the topical nail composition comprises one or more anti-infective agents, as introduced above, and a nonsteroidal anti-inflammatory drug (NSAID) agent. In further embodiments, the topical nail composition may further include urea, as also introduced above. According to various embodiments, the NSAID agent may include one or more NSAIDS selected from oxicams, such as meloxicam and piroxicam; salicylic acid derivatives, such as aspirin, diflunisal, salsalate, and trilisate; propionic acids, such as flurbiprofen, ibuprofen, ketoprofen, naproxen, or oxaprozin; acetic acids, such as diclofenac, etodolac, indomethacin, ketorolac, nabumetone, sulindac, and tolmetin; fenamates, such as meclofenamate; and/or COX-2 inhibitors, such as celecoxib, rofecoxib, and valdecoxib. All or a portion of the NSAID agent may be obtained from bulk powder. However, in some embodiments, all or a portion of the NSAID agent may be obtained from ground oral tablets, commercial solutions or suspensions, commercial NSAID topical creams such as creams, ointments, lotions, foams, solutions, gels, or combination thereof. For example, in one formula, the NSAID agent comprises the NSAID diclofenac obtained from a commercial diclofenac solution.

In various embodiments, the topical nail composition may comprise between approximately 0.01% and approximately 20% by weight NSAID, such as between approximately 0.01% and approximately 5%, between approximately 0.01% and approximately 3%, between approximately 0.01% and approximately 1%, between approximately 0.01% and approximately 0.25%, between approximately 0.01% and approximately 0.15%, or between approximately 0.05% and approximately 0.15%, between 0.1% and 10%, between approximately 0.1% and approximately 0.5%, between approximately 0.1% and approximately 0.2%, between approximately 0.2% and approximately 0.8%, between approximately 0.2% and approximately 0.6%, between approximately 0.2% and approximately 0.4%, between approximately 0.3% and approximately 1%, between approximately 0.3% and approximately 0.8%, between approximately 0.3% and approximately 0.6%, between approximately 0.4% and approximately 1%, between approximately 0.5% and approximately 1%, between approximately 0.5% and approximately 8%, between approximately 0.6% and approximately 1%, between approximately 0.6% and approximately 0.8%, between approximately 0.8% and approximately 1%, between approximately 1% and approximately 3%, between approximately 1% and approximately 10%, between approximately 1% and approximately 8%, between approximately 1% and approximately 5%, between approximately 1% and approximately 3%, between approximately 3% and approximately 10%, between approximately 3% and approximately 8%, or between approximately 3% and approximately 5%, between 5% and 10%, between approximately 5% and approximately 8%, between approximately 6% and approximately 10%, between approximately 6% and approximately 8%, between approximately 7% and approximately 10%, between approximately 8% and approximately 10%, between approximately 10% and approximately 20%, between approximately 10% and approximately 15%, between approximately 10% and approximately 12%, between approximately 12% and approximately 15%, or between approximately 15% and approximately 20% of the topical nail composition by weight. In some embodiments, the amount of NSAID by weight may be approximately 0.01%, approximately 0.05%, approximately 0.1%, approximately 0.5%, approximately 1%, approximately 1.5%, approximately 2%, approximately 2.5%, approximately 3%, approximately 3.5%, approximately 4%, approximately 4.5%, approximately 5%, approximately 5.5%, approximately 6%, approximately 6.5%, approximately 7%, approximately 7.5%, approximately 8%, approximately 8.5%, approximately 9%, approximately 9.5%, approximately 10%, approximately 11%, approximately 12%, approximately 13%, approximately 14%, approximately 15%, approximately 17%, approximately 19%, approximately 20%, or any other percentage between approximately 0.01% and 20% by weight of the topical nail composition.

The compounded nail composition may also include one or more additives as emollients thickening agents, plasticizers, polymers, volatile organic compounds, or other additives to promote ease of application, effective localization of the medication, or comfort following administration of the composition to a nail.

In some embodiments, a combination therapy for treatment of a fungal or nail infection includes sequential administration of the topical nail composition comprising one or more anti-infective agents and diluent with a DMSO and NSAID composition as disclosed herein. In one embodiment, the topical nail composition also includes urea as disclosed herein.

In some embodiments, the NSAID is combined with DMSO and used as a nail lacquer sequentially, either before or after, with the topical nail composition including the anti-infective, which may also include urea and/or diluent as described above and elsewhere herein. In one embodiment, the NSAID includes diclofenac. Diclofenac may be present in an amount between approximately 0.05% and approximately 5%, such as between approximately 1% and approximately 3%, between approximately 1% and approximately 2%, or approximately 1.5%. DMSO may be present in an amount between approximately 10% and approximately 50%, such as between approximately 15% and approximately 50%, between approximately 20% and approximately 50%, between approximately 25% and approximately 50%, between approximately 30% and approximately 50%, between approximately 35% and approximately 50%, between approximately 40% and approximately 50%, or between approximately 20% and approximately 40%, by weight.

In some embodiments, a method of treating a nail infection may include a combination treatment including applying one or more topical nail compositions described herein to nails and administration of a topical composition for footbath or irrigation administration. In one example, the topical nail composition may be applied as a pre-treatment prior to footbath or irrigation administration. The topical nail composition may comprise an NSAID and DMSO, such as disclosed herein, for example. This protocol may be followed once or twice a day. In certain further embodiments, another topical nail composition may be applied to nails between footbath or irrigation administrations. Such topical nail compositions may be any topical nail composition described herein.

### Example Topical Nail Compositions

### Example 1

One example topical nail composition comprises an antibacterial agent comprising approximately 0.6 g to approximately 1.8 g tobramycin and approximately 10 mg to approximately 50 mg mupirocin; an antifungal agent comprising approximately 20 mg to approximately 100 mg itraconazole; and approximately 100 mg to approximately 1000 mg urea per approximately 10 ml to approximately 20 ml diluent. For example, the topical nail composition may comprise approximately 1.2 g tobramycin and 20 mg mupirocin; an antifungal agent comprising approximately 50 mg itraconazole; approximately 500 mg urea; and a diluent comprising approximately 15 mL 0.09% Sterile Sodium Chloride. The composition may be applied to nails once daily or as otherwise directed, such as two or more times a day. In various embodiments, the tobramycin comprises Tobramycin for Injection, USP. The tobramycin may also include other tobramycin formats, such as those described herein. The mupirocin may include a bulk powder, ground tablets, or a commercially available mupirocin ointment or cream. The itraconazole may be a bulk powder, capsule, ground tablet, or other commercially available itraconazole format. The urea may be a bulk urea powder, ointment, or cream. In various embodiments, the amount of diluent may be reduced by an amount of base carrier provided by the anti-infective agents or urea agent. In various embodiments, the tobramycin may be replaced or supplemented with voriconazole, colistimethate, or ceftriaxone, such as approximately 200 mg Voriconazole for Injection, approximately 300 mg Colistimethate for Injection, or approximately 500 mg Ceftriaxone for Injection. In some embodiments, the topical nail composition may include or may be administered in combination with a topical nail composition including an NSAID comprising one or more NSAIDs and DMSO. For example, an NSAID agent and DMSO as disclosed herein may be combined with the topical nail composition comprising an anti-infective or may be administered separate as part of a combination treatment. In some embodiments, the NSAID comprises diclofenac, e.g., approximately 1.5% diclofenac and approximately 45.5% DMSO. The NSAID and DMSO may be provided in a 10 mL solution. In one example, the solution of DMSO and NSAID replace all or a portion of the sodium chloride. The DMSO and NSAID solution may be applied prior to administration of the topical nail composition comprising the anti-infectives. In some embodiments wherein the DMSO and NSAID are combined with the topical nail composition comprising anti-infectives, the amount of diluent may be reduced by an amount of base carrier provided by an anti-infective agent, urea agent, NSAID agent, or DMSO. The composition may be applied to nails in a thin layer once daily or as otherwise directed, such as two or more times a day. The composition may typically be applied to nails in a thin layer just prior to administration of the topical nail composition comprising the anti-infectives.

### Example 2

Another example topical nail composition comprises an antibacterial agent comprising approximately 40 mg to approximately 120 mg gentamicin and approximately 10 mg to approximately 50 mg mupirocin; an antifungal agent comprising approximately 20 mg to approximately 100 mg itraconazole; and approximately 100 mg to approximately 1000 mg urea per approximately 10 ml to approximately 20 ml diluent. For example, the topical nail composition may comprise approximately 80 mg gentamicin, approximately 20 mg mupirocin, approximately 50 mg itraconazole, approximately 500 mg urea, and a diluent comprising approximately 15 mL 0.09% Sterile Sodium Chloride. The composition may be applied to nails once daily or as otherwise directed, such as two or more times a day. In various embodiments, the gentamicin comprises gentamicin bulk powder, injection solution, or ointment. The mupirocin may include a bulk powder, ground tablets, or a commercially available mupirocin ointment or cream, for example. The itraconazole may be a bulk powder, capsule, ground tablet, or other commercially available itraconazole format. The urea may be a bulk urea powder, ointment, or cream. In various embodiments, the amount of diluent may be reduced by an amount of base carrier provided by the anti-infective agents or urea agent. In various embodiments, the gentamicin may be replaced or supplemented with voriconazole, tobramycin, colistimethate, or ceftriaxone, such as approximately 200 mg Voriconazole for Injection, approximately 1.2 g Tobramycin for Injection, approximately 300 mg Colistimethate for Injection, or approximately 500 mg Ceftriaxone for Injection. In some embodiments, the topical nail composition may include or may be administered in combination with a topical nail composition including an NSAID comprising one or more NSAIDs and DMSO. For example, an NSAID agent and DMSO as disclosed herein may be combined with the topical nail composition comprising an anti-infective or may be administered separate as part of a combination treatment. In some embodiments, the NSAID comprises diclofenac, e.g., approximately 1.5% diclofenac and approximately 45.5% DMSO. The NSAID and DMSO may be provided in a 10 mL solution. In one example, the solution of DMSO and NSAID replace all or a portion of the sodium chloride. The DMSO and NSAID solution may be applied prior to administration of the topical nail composition comprising the anti-infectives. In some embodiments wherein the DMSO and NSAID are combined with the topical nail composition comprising anti-infectives, the amount of diluent may be reduced by an amount of base carrier provided by an anti-infective agent, urea agent, NSAID agent, or DMSO. The composition may be applied to nails in a thin layer once daily or as otherwise directed, such as two or more times a day. The composition may typically be applied to nails in a thin layer just prior to administration of the topical nail composition comprising the anti-infectives.

### Example 3

Another example topical nail composition comprises an antibacterial agent comprising approximately 100 mg to approximately 400 mg colistimethate and approximately 10 mg to approximately 50 mg mupirocin; an antifungal agent comprising approximately 20 mg to approximately 100 mg itraconazole; and approximately 100 mg to approximately 1000 mg urea per approximately 10 ml to approximately 20 ml diluent. For example, the topical nail composition may comprise approximately 300 mg colistimethate, approximately 20 mg mupirocin, approximately 50 mg itraconazole, approximately 500 mg urea, and a diluent comprising approximately 15 mL 0.09% Sterile Sodium Chloride. The composition may be applied to nails once daily or as otherwise directed, such as two or more times a day. In various embodiments, the colistimethate comprises colistimethate bulk powder, injection solution, or ointment. The mupirocin may include a bulk powder, ground tablets, or a commercially available mupirocin ointment or cream, for example. The itraconazole may be a bulk powder, capsule, ground tablet, or other commercially available itraconazole format. The urea may be a bulk urea powder, ointment, or cream. In various embodiments, the amount of diluent may be reduced by an amount of base carrier provided by an anti-infective agent or urea agent. In various embodiments, the colistimethate may be replaced or supplemented with voriconazole, tobramycin, gentamicin, or ceftriaxone, such as approximately 200 mg Voriconazole for Injection, approximately 1.2 g Tobramycin for Injection, approximately 100 mg gentamycin, or approximately 500 mg Ceftriaxone for Injection. In some embodiments, the topical nail composition may include or may be administered in combination with a topical nail composition including an NSAID comprising one or more NSAIDs and DMSO. For example, an NSAID agent and DMSO as disclosed herein may be combined with the topical nail composition comprising an anti-infective or may be administered separate as part of a combination treatment. In some embodiments, the NSAID comprises diclofenac, e.g., approximately 1.5% diclofenac and approximately 45.5% DMSO. The NSAID and DMSO may be provided in a 10 mL solution. In one example, the solution of DMSO and NSAID replace all or a portion of the sodium chloride. The DMSO and NSAID solution may be applied prior to administration of the topical nail composition comprising the anti-infectives. In some embodiments wherein the DMSO and NSAID are combined with the topical nail composition comprising anti-infectives, the amount of diluent may be reduced by an amount of base carrier provided by an anti-infective agent, urea agent, NSAID agent, or DMSO. The composition may be applied to nails in a thin layer once daily or as otherwise directed, such as two or more times a day. The composition may typically be applied to nails in a thin layer just prior to administration of the topical nail composition comprising the anti-infectives.

### Example 4

Another example topical nail composition comprises an antifungal agent comprising approximately 100 mg to approximately 300 mg voriconazole; and approximately 100 mg to approximately 1000 mg urea per approximately 10 ml to approximately 20 ml diluent. For example, the topical nail composition may comprise approximately 200 mg voriconazole; approximately 500 mg urea; and a diluent comprising approximately 15 mL 0.09% Sterile Sodium Chloride. The composition may be applied to nails once daily or as otherwise directed, such as two or more times a day. In various embodiments, the voriconazole comprises gentamicin bulk powder, for injection, injection solution, ointment, or other commercially available voriconazole format. The urea may be a bulk urea powder, ointment, or cream. In various embodiments, the amount of diluent may be reduced by an amount of base carrier provided by the anti-infective agent or urea agent. In various embodiments, the voriconazole may be replaced by tobramycin for injection, colistimethate, or ceftriaxone, such as approximately 1.2 g Tobramycin for Injection, approximately 300 mg Colistimethate for Injection, or approximately 500 mg Ceftriaxone for Injection. In some embodiments, the topical nail composition may include or may be administered in combination with a topical nail composition including an NSAID comprising one or more NSAIDs and DMSO. For example, an NSAID agent and DMSO as disclosed herein may be combined with the topical nail composition comprising an anti-infective or may be administered separate as part of a combination treatment. In some embodiments, the NSAID comprises diclofenac, e.g., approximately 1.5% diclofenac and approximately 45.5% DMSO. The NSAID and DMSO may be provided in a 10 mL solution. In one example, the solution of DMSO and NSAID replace all or a portion of the sodium chloride. The DMSO and NSAID solution may be applied prior to administration of the topical nail composition comprising the anti-infectives. In some embodiments wherein the DMSO and NSAID are combined with the topical nail composition comprising anti-infectives, the amount of diluent may be reduced by an amount of base carrier provided by an anti-infective agent, urea agent, NSAID agent, or DMSO. The composition may be applied to nails in a thin layer once daily or as otherwise directed, such as two or more times a day. The composition may typically be applied to nails in a thin layer just prior to administration of the topical nail composition comprising the anti-infectives.

### Example 5

Another example topical nail composition comprises an antifungal agent comprising approximately 50 mg to approximately 200 mg itraconazole; and approximately 100 mg to approximately 1000 mg urea per approximately 10 ml to approximately 20 ml diluent. For example, the topical nail composition may comprise approximately 100 mg itraconazole; approximately 500 mg urea; and a diluent comprising approximately 15 mL 0.09% Sterile Sodium Chloride. The composition may be applied to nails once daily or as otherwise directed, such as two or more times a day. In various embodiments, the itraconazole may be a bulk powder, capsule, ground tablet, or other commercially available itraconazole format. The urea may be a bulk urea powder, ointment, or cream. In various embodiments, the amount of diluent may be reduced by an amount of base carrier provided by the anti-infective agent or urea agent. In various embodiments, the itraconazole may be replaced or supplemented with voriconazole, tobramycin, colistimethate, or ceftriaxone, such as approximately 200 mg Voriconazole for Injection, approximately 1.2 g Tobramycin for Injection, approximately 300 mg Colistimethate for Injection, or approximately 500 mg Ceftriaxone for Injection. In some embodiments, the topical nail composition may include or may be administered in combination with a topical nail composition including an NSAID comprising one or more NSAIDs and DMSO. For example, an NSAID agent and DMSO as disclosed herein may be combined with the topical nail composition comprising an anti-infective or may be administered separate as part of a combination treatment. In some embodiments, the NSAID comprises diclofenac, e.g., approximately 1.5% diclofenac and approximately 45.5% DMSO. The NSAID and DMSO may be provided in a 10 mL solution. In one example, the solution of DMSO and NSAID replace all or a portion of the sodium chloride. The DMSO and NSAID solution may be applied prior to administration of the topical nail composition comprising the anti-infectives. In some embodiments wherein the DMSO and NSAID are combined with the topical nail composition comprising anti-infectives, the amount of diluent may be reduced by an amount of base carrier provided by an anti-infective agent, urea agent, NSAID agent, or DMSO. The composition may be applied to nails in a thin layer once daily or as otherwise directed, such as two or more times a day. The composition may typically be applied to nails in a thin layer just prior to administration of the topical nail composition comprising the anti-infectives.

### Example 6

Another example topical nail composition comprises an antibacterial agent comprising approximately 500 mg to approximately 1000 mg tetracycline per approximately 10 ml to approximately 20 ml diluent. For example, the topical nail composition may comprise approximately 1000 mg tetracycline and approximately 15 mL 0.09% Sterile Sodium Chloride. The composition may be applied to nails once daily or as otherwise directed, such as two or more times a day. In various embodiments, the tetracycline may be a bulk powder, capsule, ground tablet, or other commercially available tetracycline format. In various embodiments, the amount of diluent may be reduced by an amount of base carrier provided by the anti-infective agent. In various embodiments, the tetracycline may be replaced or supplemented with voriconazole, tobramycin, colistimethate, or ceftriaxone, such as approximately 200 mg Voriconazole for Injection, approximately 1.2 g Tobramycin for Injection, approximately 300 mg Colistimethate for Injection, or approximately 500 mg Ceftriaxone for Injection. In some embodiments, the topical nail composition may also include approximately 100 mg to approximately 1000 mg urea, e.g., approximately 500 mg urea. In some embodiments, the topical nail composition may include or may be administered in combination with a topical nail composition including an NSAID comprising one or more NSAIDs and DMSO. For example, an NSAID agent and DMSO as disclosed herein may be combined with the topical nail composition comprising an anti-infective or may be administered separate as part of a combination treatment. In some embodiments, the NSAID comprises diclofenac, e.g., approximately 1.5% diclofenac and approximately 45.5% DMSO. The NSAID and DMSO may be provided in a 10 mL solution. In one example, the solution of DMSO and NSAID replace all or a portion of the sodium chloride. The DMSO and NSAID solution may be applied prior to administration of the topical nail composition comprising the anti-infectives. In some embodiments wherein the DMSO and NSAID are combined with the topical nail composition comprising anti-infectives, the amount of diluent may be reduced by an amount of base carrier provided by the anti-infective agent, NSAID agent, or DMSO. The composition may be applied to nails in a thin layer once daily or as otherwise directed, such as two or more times a day. The composition may typically be applied to nails in a thin layer just prior to administration of the topical nail composition comprising the anti-infectives.

### Example 7

Another example topical nail composition comprises an antibacterial agent comprising approximately 10 mg to approximately 50 mg mupirocin; and an antifungal agent comprising approximately 20 mg to approximately 100 mg itraconazole per approximately 10 ml to approximately 20 ml diluent. For example, the topical nail composition may comprise approximately 20 mg mupirocin, approximately 50 mg itraconazole, and a diluent comprising approximately 15 mL 0.09% Sterile Sodium Chloride. The composition may be applied to nails once daily or as otherwise directed, such as two or more times a day. The mupirocin may include a bulk powder, ground tablets, or a commercially available mupirocin ointment or cream, for example. The itraconazole may be a bulk powder, capsule, ground tablet, or other commercially available itraconazole format. In various embodiments, the amount of diluent may be reduced by an amount of base carrier provided by the anti-infective agents. In some embodiments, the topical nail composition may also include approximately 100 mg to approximately 1000 mg urea, e.g., approximately 500 mg urea. In some embodiments, the topical nail composition may include or may be administered in combination with a topical nail composition including an NSAID comprising one or more NSAIDs and DMSO. For example, an NSAID agent and DMSO as disclosed herein may be combined with the topical nail composition comprising an anti-infective or may be administered separate as part of a combination treatment. In some embodiments, the NSAID comprises diclofenac, e.g., approximately 1.5% diclofenac and approximately 45.5% DMSO. The NSAID and DMSO may be provided in a 10 mL solution. In one example, the solution of DMSO and NSAID replace all or a portion of the sodium chloride. The DMSO and NSAID solution may be applied prior to administration of the topical nail composition comprising the anti-infectives. In some embodiments wherein the DMSO and NSAID are combined with the topical nail composition comprising anti-infectives, the amount of diluent may be reduced by an amount of base carrier provided by an anti-infective agent, NSAID agent, or DMSO. The composition may be applied to nails in a thin layer once daily or as otherwise directed, such as two or more times a day. The composition may typically be applied to nails in a thin layer just prior to administration of the topical nail composition comprising the anti-infectives.

### Example 8

Another example topical nail composition comprises an antibacterial agent comprising approximately 50 mg to approximately 200 mg doxycycline and approximately 10 mg to approximately 50 mg mupirocin; and an antifungal agent comprising approximately 15 mg to approximately 50 mg clotrimazole per approximately 10 ml to approximately 20 ml diluent. For example, the topical nail composition may comprise approximately 100 mg doxycycline, approximately 30 mg mupirocin, approximately 30 mg clotrimazole, and a diluent comprising approximately 15 mL 0.09% Sterile Sodium Chloride. The composition may be applied to nails once daily or as otherwise directed, such as two or more times a day. The doxycycline may be a bulk powder, capsule, ground tablet, or other commercially available doxycycline format. The mupirocin may include a bulk powder, ground tablets, or a commercially available mupirocin ointment or cream, for example. The clotrimazole may be a bulk powder, capsule, ground tablet, cream, ointment, or other commercially available clotrimazole format. In various embodiments, the amount of diluent may be reduced by an amount of base carrier provided by the anti-infective agents. In some embodiments, the topical nail composition may also include approximately 100 mg to approximately 1000 mg urea, e.g., approximately 500 mg urea. In some embodiments, the topical nail composition may include or may be administered in combination with a topical nail composition including an NSAID comprising one or more NSAIDs and DMSO. For example, an NSAID agent and DMSO as disclosed herein may be combined with the topical nail composition comprising an anti-infective or may be administered separate as part of a combination treatment. In some embodiments, the NSAID comprises diclofenac, e.g., approximately 1.5% diclofenac and approximately 45.5% DMSO. The NSAID and DMSO may be provided in a 10 mL solution. In one example, the solution of DMSO and NSAID replace all or a portion of the sodium chloride. The DMSO and NSAID solution may be applied prior to administration of the topical nail composition comprising the anti-infectives. In some embodiments wherein the DMSO and NSAID are combined with the topical nail composition comprising anti-infectives, the amount of diluent may be reduced by an amount of base carrier provided by an anti-infective agent, NSAID agent, or DMSO. The composition may be applied to nails in a thin layer once daily or as otherwise directed, such as two or more times a day. The composition may typically be applied to nails in a thin layer just prior to administration of the topical nail composition comprising the anti-infectives.

### Example 9

Another example topical nail composition comprises an anti-infective agent comprising approximately 10 mg to approximately 100 mg nitrofurantoin per approximately 10 ml to approximately 20 ml diluent. For example, the topical nail composition may comprise approximately 50 mg nitrofurantoin and a diluent comprising approximately 15 mL 0.09% Sterile Sodium Chloride. The composition may be applied to nails once daily or as otherwise directed, such as two or more times a day. The nitrofurantoin may include a bulk powder, solution, ground tablets, or a commercially available nitrofurantoin ointment or cream, for example. In various embodiments, the amount of diluent may be reduced by an amount of base carrier provided by the anti-infective agent. In some embodiments, the topical nail composition may also include approximately 100 mg to approximately 1000 mg urea, e.g., approximately 500 mg urea. In some embodiments, the topical nail composition may include or may be administered in combination with a topical nail composition including an NSAID comprising one or more NSAIDs and DMSO. For example, an NSAID agent and DMSO as disclosed herein may be combined with the topical nail composition comprising an anti-infective or may be administered separate as part of a combination treatment. In some embodiments, the NSAID comprises diclofenac, e.g., approximately 1.5% diclofenac and approximately 45.5% DMSO. The NSAID and DMSO may be provided in a 10 mL solution. In one example, the solution of DMSO and NSAID replace all or a portion of the sodium chloride. The DMSO and NSAID solution may be applied prior to administration of the topical nail composition comprising the anti-infectives. In some embodiments wherein the DMSO and NSAID are combined with the topical nail composition comprising anti-infectives, the amount of diluent may be reduced by an amount of base carrier provided by an anti-infective agent, NSAID agent, or DMSO. The composition may be applied to nails in a thin layer once daily or as otherwise directed, such as two or more times a day. The composition may typically be applied to nails in a thin layer just prior to administration of the topical nail composition comprising the anti-infectives.

### Methods of Making A Topical Nail Composition

A method of making a topical nail composition may comprise combining an anti-infective agent and diluent. Combining may include mixing the components to formulate a combined solution, suspension, mixture, or emulsion. In various embodiments, the method may include combining anti-infective agents comprising an antibacterial agent, an antifungal agent, or both, and a diluent. The antibacterial agent may comprise one or more antibacterials, such as those described herein. The antifungal agent may comprise one or more antifungals, such as those described herein. The diluent may comprise a diluent as disclosed herein, which may include diluent comprising carriers, which may include base vehicles, from the active agent components combined during the method. The method may include combining urea. The method may also include combining an NSAID agent. The method may also include combining DMSO.

In various embodiments, making the topical nail composition comprises encapsulating or providing one or more of the anti-infective agents into one or more capsules. The capsules may be opened to release the contents when formulating the topical nail composition. The anti-infective agents may be obtained from bulk powder or ground tablets. The one or more capsules may be provided in a kit for formulation with other components of the topical nail composition prior to administration. For example, a kit may include one more capsules of anti-infective agent powders, such as commercially available bulk powders, ground oral tablets, vials containing anti-infective powder, solution, or suspensions, which may include commercially available parenteral, injection, oral, ophthalmic, otic, or skin anti-infective compositions; tubes or containers containing anti-infective agent topical creams, gels, ointments, lotions, emulsions, foams, which may include commercially available parenteral, injection, oral, ophthalmic, otic, or skin anti-infective compositions. In one example, making the topical nail composition comprises encapsulating the anti-infective agent powders obtained from bulk powders into one or more compounded capsules comprising the amount needed or a divisible amount needed to formulate the topical nail composition. For example, in an embodiment requiring tobramycin, itraconazole, mupirocin, and urea. Making the topical nail composition may comprise encapsulating itraconazole and mupirocin bulk powders, encapsulating urea bulk powder, and providing a vial of Tobramycin for Injection. An example capsule containing itraconazole and mupirocin may include itraconazole and mupirocin in a ratio between approximately 1:20 and approximately 20:1. In one embodiment, a capsule includes mupirocin and itraconazole in a ratio of approximately 2:5. For example, a compounded capsule may include approximately 20 mg of Mupirocin USP and approximately 30 mg of Itraconazole USP Micronized. Capsules may comprise mupirocin, doxycycline, and clotrimazole. Capsules may include nitrofurantoin separately or combined with other anti-infectives and/or excipients. Capsules may include tetracycline separately or combined with other anti-infectives and/or excipients. Capsules may include colistimethate separately or combined with other anti-infectives and/or excipients. Capsules may include gentamicin separately or combined with other anti-infectives and/or excipients. Capsules may include itraconazole separately or combined with other anti-infectives and/or excipients. As introduced above, capsules containing anti-infective agents may also include excipients. Excipients may include dispersants, diluents, or surfactants, for example. Excipients may include xylitol or poloxamers. In one embodiment, a capsule containing anti-infective agent powders may include PCCA LoxaSperse™. LoxaSperse™ is an excipient base powder manufactured by PCCA (Houston, Tex.) used as a chemical dispersing or solubilizing agent in irrigation or nebulization formulations, improving the solubility and dispersibility of poorly water soluble Active Pharmaceutical Ingredients (APIs) or agents. LoxaSperse is a blend of specially micronized xylitol with an optimized ratio of micronized poloxamers, designed to improve the dispersibility and solubility of APIs. In some embodiments, XyliFos™ excipient base powder comprising xylitol, poloxamer 407, hydroxylpropyl betadex, and epigallocatechin gallate may be used. LoxaSperse™, XyliFos™, or both may be used. The ratio of LoxaSperse™ or XyliFos™ to anti-infective agent in a capsule may be approximately 20:1 and approximately 1:20. For example, in one embodiment, a capsule compounded to contain approximately 20 mg of Mupirocin USP and approximately 30 mg of Itraconazole USP Micronized, may further contain approximately 276 mg LoxaSperse™.

In one embodiment, urea may be provided in a capsule comprising urea powder. The capsule may be opened to release the contents when formulating the topical nail composition. The urea powder may be obtained from bulk urea USP powder. The capsule may include additional actives or inactives. In some embodiments, a capsule containing urea may include an excipient. Excipients may include dispersants, diluents, or surfactants, for example. Excipients may include xylitol or poloxamers. In one embodiment, a capsule containing urea may include PCCA LoxaSperse™. LoxaSperse™ is an excipient base powder manufactured by PCCA (Houston, Tex.) used as a chemical dispersing or solubilizing agent in irrigation or nebulization formulations, improving the solubility and dispersibility of poorly water soluble Active Pharmaceutical Ingredients (APIs) or agents. LoxaSperse is a blend of specially micronized xylitol with an optimized ratio of micronized poloxamers, designed to improve the dispersibility and solubility of APIs. In some embodiments, XyliFos™ excipient base powder comprising xylitol, poloxamer 407, hydroxylpropyl betadex, and epigallocatechin gallate may be used.

In one embodiment, making the topical nail composition comprises combining urea powder with an excipient base comprising LoxaSperse™ to form the dry powder formulation. Encapsulating the dry powder may comprise encapsulating the LoxaSperse and approximately 500 mg of urea or other amount needed for the formulation, preferably in an amount that the need amount is divisible by, in the capsule. The capsule may contain LoxaSperse™ in a ratio of approximately 1:4 with respect to urea, such as approximately 131 mg in a capsule containing 500 mg urea. Other ratios may be used, e.g., between approximately 20:1 and approximately 1:20. Making the topical nail composition may further comprise obtaining the urea from a bulk source.

In some embodiments, making the topical nail composition may include providing a vial or container containing DMSO. The vial or container may also include an NSAID. For example, the vial or container may include DMSO and diclofenac. DMSO makes up approximately 45.5% of the diclofenac solution (1.5% Stock Solution). The DMSO may be included in a kit for the treatment of a fungal or bacterial nail infection. In a further example, making the topical nail composition may include providing a vial or container containing an NSAID solution or suspension. For example, diclofenac may be provided in an aqueous solution comprising a diclofenac sodium solution. In one embodiment, the diclofenac or pharmaceutically acceptable salt thereof may comprise a diclofenac sodium solution for topical application. The diclofenac sodium solution may contain, for example, 1.5% (w/w) diclofenac sodium wherein each 1 mL of solution may contain approximately 16.05 mg of diclofenac sodium. In one embodiment, the diclofenac solution comprises a diclofenac sodium solution 1.5% (w/w) such as that manufactured under the trade name PENNSAID® by Nuvo Manufacturing, Varennes, Quebec, Canada for treating the pain of osteoarthritis of the knee. The diclofenac solution may also contain various inactive ingredients such as dimethyl sulfoxide USP (DMSO, 45.5% w/w), ethanol, glycerin, propylene glycol and purified water. In one embodiment, the diclofenac solution comprises a diclofenac sodium solution marketed under the trade name PENNSAID® and manufactured by Nuvo Manufacturing, Varennes, Quebec, Canada, in a 2% (w/w) diclofenac solution for treating the pain of osteoarthritis of the knee. Each gram of solution may contain approximately 20 mg of diclofenac sodium and various inactive ingredients such as dimethyl sulfoxide USP (DMSO, 45.5% w/w), ethanol, purified water, propylene glycol, and hydroxypropyl cellulose. In other embodiments, other concentrations of diclofenac solution, such as diclofenac sodium solutions, may be used. In another example, making the topical nail composition comprises encapsulating or providing a capsule containing an NSAID powder, e.g., bulk powder or ground tablet. Such vials, containers, or capsules containing DMSO, NSAID, or both may be provided in a kit for the treatment of a bacterial or fungal nail infection.

Making the topical nail composition may comprise adding the contents of one or more capsules, vials, tubes or containers containing the components of the composition and mixing to form the topical nail composition.

In one example, a method of making a topical nail composition comprises combining an antibacterial agent comprising between approximately 0.06 g and approximately 1.5 g of a first antibacterial, an antifungal agent comprising between approximately 0.01 g and approximately 1.5 g of a first antifungal, or both, and between approximately 5 mL and approximately 25 mL of a diluent. The first antibacterial or first antifungal may be any suitable antibacterial or antifungal, such as those described herein. In various embodiments, at least one of the first antibacterial or first antifungal comprises an antibacterial or antifungal for injection. It will be understood that the example formulations herein may be suitably scaled for making larger or smaller batches of topical nail composition. In some embodiments, the antibacterial agent, antifungal agent, or both may include a second antibacterial or antifungal. The second antibacterial or antifungal may be added in an amount between approximately 0.01 g and approximately 0.5 g. The second antibacterial or second antifungal may be any suitable antibacterial or antifungal, such as those described herein. The method may also include combining between approximately 0.1 g and 1.5 g urea. In a further example method, the method includes combining an NSAID agent comprising one or more NSAIDs, such as those described herein. The NSAID may be added in an amount between approximately 0.01 g to approximately 1 g. In a further example of the method, the method comprises combining DMSO. DMSO may be added in a solution to combine with one or more additional components. The DMSO may be added in an amount between approximately 1 g and approximately 10 g. The diluent may be adjusted to obtain a final volume of the topical nail composition between approximately 6 mL and approximately 25 mL. It also be appreciated that the example methods are not limited to an order of combining the various components.

In one example, a method of making a topical nail composition comprises combining an antibacterial agent comprising approximately 1.2 g Tobramycin for Injection, USP and approximately 20 mg mupirocin bulk powder; an antifungal agent comprising approximately 50 mg itraconazole bulk powder; approximately 500 mg urea bulk powder; and a diluent comprising approximately 15 mL 0.09% Sterile Sodium Chloride. In various embodiments, the tobramycin may be replaced or supplemented with voriconazole, colistimethate, or ceftriaxone, such as approximately 200 mg Voriconazole for Injection, approximately 300 mg Colistimethate for Injection, or approximately 500 mg Ceftriaxone for Injection. A method of treating a bacterial or fungal nail infection may comprise applying the composition to an affected nail once daily or as otherwise directed, such as two or more times a day.

In another example, a method of making a topical nail composition comprises combining an antibacterial agent comprising approximately 80 mg gentamicin bulk powder and approximately 20 mg mupirocin bulk powder; an antifungal agent comprising approximately 50 mg itraconazole bulk powder; approximately 500 mg urea; and a diluent comprising approximately 15 mL 0.09% Sterile Sodium Chloride. In various embodiments, the gentamicin may be replaced or supplemented with voriconazole, tobramycin, colistimethate, or ceftriaxone, such as approximately 200 mg Voriconazole for Injection, approximately 1.2 g Tobramycin for Injection, approximately 300 mg Colistimethate for Injection, or approximately 500 mg Ceftriaxone for Injection. A method of treating a bacterial or fungal nail infection may comprise applying the composition to an affected nail once daily or as otherwise directed, such as two or more times a day.

According to another example, a method of making a topical nail composition comprises combining an antifungal agent comprising approximately 200 mg Voriconazole for Injection; approximately 500 mg urea; and a diluent comprising approximately 15 mL 0.09% Sterile Sodium Chloride. A method of treating a bacterial or fungal nail infection may comprise applying the composition to an affected nail once daily or as otherwise directed, such as two or more times a day. In various embodiments, the voriconazole may be replaced or supplemented with tobramycin, colistimethate, or ceftriaxone, such as approximately 1.2 g Tobramycin for Injection, approximately 300 mg Colistimethate for Injection, or approximately 500 mg Ceftriaxone for Injection.

According to another example, a method of making a topical nail composition comprises combining an antifungal agent comprising approximately 100 mg itraconazole bulk powder; approximately 500 mg urea bulk powder; and a diluent comprising approximately 15 mL 0.09% Sterile Sodium Chloride. A method of treating a bacterial or fungal nail infection may comprise applying the composition to an affected nail once daily or as otherwise directed, such as two or more times a day. In various embodiments, the itraconazole may be replaced or supplemented with voriconazole, tobramycin, colistimethate, or ceftriaxone, such as approximately 200 mg Voriconazole for Injection, approximately 1.2 g Tobramycin for Injection, approximately 300 mg Colistimethate for Injection, or approximately 500 mg Ceftriaxone for Injection.

According to another example, a method of making a topical nail composition comprises combining an antibacterial agent comprising approximately 1.2 g Tobramycin for Injection, USP, and approximately 20 mg mupirocin bulk powder; an antifungal agent comprising approximately 50 mg itraconazole bulk powder; approximately 500 mg urea; approximately 10 mL solution comprising approximately 1.5% diclofenac and approximately 45.5% DMSO; and a diluent comprising approximately 15 mL 0.09% Sterile Sodium Chloride. In various embodiments, the tobramycin may be replaced or supplemented with voriconazole, colistimethate, or ceftriaxone, such as approximately 200 mg Voriconazole for Injection, approximately 300 mg Colistimethate for Injection, or approximately 500 mg Ceftriaxone for Injection. In some embodiments, the amount of diluent may be optionally reduced to less than approximately 10 mL, less than approximately 7 mL, less than approximately 5 mL, less than approximately 3 mL, or excluded. For example, the solution of DMSO and NSAID may replace all or a portion of the sodium chloride. A method of treating a bacterial or fungal nail infection may comprise applying the composition to an affected nail once daily or as otherwise directed, such as two or more times a day.

According to another example, a method of making a topical nail composition comprises combining an antibacterial agent comprising approximately 80 mg gentamicin bulk powder and approximately 20 mg mupirocin bulk powder; an antifungal agent comprising approximately 50 mg itraconazole bulk powder; approximately 500 mg urea; approximately 10 mL solution comprising approximately 1.5% diclofenac and approximately 45.5% DMSO; and a diluent comprising approximately 15 mL 0.09% Sterile Sodium Chloride. In various embodiments, the gentamicin may be replaced or supplemented with voriconazole, tobramycin, colistimethate, or ceftriaxone, such as approximately 200 mg Voriconazole for Injection, approximately 1.2 g Tobramycin for Injection, approximately 300 mg Colistimethate for Injection, or approximately 500 mg Ceftriaxone for Injection. In some embodiments, the amount of diluent may be optionally reduced to less than approximately 10 mL, less than approximately 7 mL, less than approximately 5 mL, less than approximately 3 mL, or excluded. For example, the solution of DMSO and NSAID may replace all or a portion of the sodium chloride. A method of treating a bacterial or fungal nail infection may comprise applying the composition to an affected nail once daily or as otherwise directed, such as two or more times a day. In various embodiments, the gentamicin may be replaced or supplemented with voriconazole, tobramycin for injection, colistimethate, or ceftriaxone, such as approximately 200 mg Voriconazole for Injection, approximately 1.2 g Tobramycin for Injection, approximately 300 mg Colistimethate for Injection, or approximately 500 mg Ceftriaxone for Injection.

According to another example, a method of making a topical nail composition comprises combining an antifungal agent comprising approximately 200 mg Voriconazole for Injection; approximately 500 mg urea bulk powder; approximately 10 mL solution comprising approximately 1.5% diclofenac and approximately 45.5% DMSO; and a diluent comprising approximately 15 mL 0.09% Sterile Sodium Chloride. In various embodiments, the voriconazole may be replaced or supplemented with tobramycin, colistimethate, or ceftriaxone, such as approximately 1.2 g Tobramycin for Injection, approximately 300 mg Colistimethate for Injection, or approximately 500 mg Ceftriaxone for Injection. In some embodiments, the amount of diluent may be optionally reduced to less than approximately 10 mL, less than approximately 7 mL, less than approximately 5 mL, less than approximately 3 mL, or excluded. For example, the solution of DMSO and NSAID may replace all or a portion of the sodium chloride. A method of treating a bacterial or fungal nail infection may comprise applying the composition to an affected nail once daily or as otherwise directed, such as two or more times a day. In various embodiments, the voriconazole may be replaced by tobramycin for injection, colistimethate, or ceftriaxone, such as approximately 1.2 g Tobramycin for Injection, approximately 300 mg Colistimethate for Injection, or approximately 500 mg Ceftriaxone for Injection.

According to another example, a method of making a topical nail composition comprises combining an antifungal agent comprising approximately 100 mg itraconazole; approximately 500 mg urea; approximately 10 mL solution comprising approximately 1.5% diclofenac and approximately 45.5% DMSO; and a diluent comprising approximately 15 mL 0.09% Sterile Sodium Chloride. In various embodiments, the itraconazole may be replaced or supplemented with voriconazole, tobramycin, colistimethate, or ceftriaxone, such as approximately 200 mg Voriconazole for Injection, approximately 1.2 g Tobramycin for Injection, approximately 300 mg Colistimethate for Injection, or approximately 500 mg Ceftriaxone for Injection. In some embodiments, the amount of diluent may be optionally reduced to less than approximately 10 mL, less than approximately 7 mL, less than approximately 5 mL, less than approximately 3 mL, or excluded. For example, the solution of DMSO and NSAID may replace all or a portion of the sodium chloride. A method of treating a bacterial or fungal nail infection may comprise applying the composition to an affected nail once daily or as otherwise directed, such as two or more times a day.

### Topical Compositions

Topical compositions described herein include formulations for administration by irrigation or within a footbath. The topical compositions may be used alone or as part of a combination treatment to treat a nail infection or suspected nail infection of the hand or foot. In some examples, the topical composition may be administered to treat a microbial infection of the foot or hand together with a nail infection alone or in combination with one or more additional topical compositions and/or one or more topical nail compositions.

In various embodiments, a topical composition, which may also be referred to as a footbath formulation or dosage form, to treat of a bacterial or fungal nail infection comprises one or more anti-infective agents and a diluent. The anti-infective agent may include one or more antibacterial agents, antifungal agents, antiviral agents, such as pharmaceutical drug antibiotics, antifungals, and antivirals, or combinations thereof.

In various embodiments, an antibacterial agent may include one or more enicillins, cephalosporins, fluoroquinolones, aminoglycosides, monobactams, carbapenems, macrolides, and other antibacterials. For example, the antibacterial agent may include one or more antibacterials selected from afenide, amikacin, amoxicillin, ampicillin, arsphenamine, azithromycin, azlocillin, aztreonam, bacampicillin, bacitracin, carbacephem (loracarbef), carbenicillin, cefaclor, cefadroxil, cefalotin, cefamandole, cefazolin, cefdinir, cefditoren, cefepime, cefixime, cefoperazone, cefotaxime, cefoxitin, cefpodoxime, cefprozil, ceftazidime, ceftibuten, ceftizoxime, ceftobiprole, ceftriaxone, cefuroxime, cephalexin, chloramphenicol, chlorhexidine, ciprofloxacin, clarithromycin, clavulanic acid, clindamycin, cloxacillin, colimycin, colistimethate teicoplanin, colistin, demeclocycline, dicloxacillin, dirithromycin, doripenem, doxycycline, efprozil, enoxacin, ertapenem, erythromycin, ethambutol, flucloxacillin, fosfomycin, furazolidone, gatifloxacin, geldanamycin, gentamicin, grepafloxacin, herbimycin, imipenem, isoniazid, kanamycin, levofloxacin, lincomycin, linezolid, lomefloxacin, meropenem, meticillin, meticillin, mezlocillin, minocycline, mitomycin, moxifloxacin, mupirocin, nafcillin, neomycin, netilmicin, nitrofurantoin, norfloxacin, ofloxacin, oxacillin, oxytetracycline, paromomycin, penicillin G, penicillin V, piperacillin, pivmecillinam, platensimycin, polymyxin B, prontosil, pvampicillin, pyrazinamide, quinupristin/dalfopristin, rifampicin, rifampin, roxithromycin, sparfloxacin, spectinomycin, spiramycin, sulbactam, sulfacetamide, sulfamethizole, sulfamethoxazole, sulfanilimide, sulfisoxazole, sulphonamides, sultamicillin, telithromycin, tetracycline, thiamphenicol, ticarcillin, tobramycin, trimethoprim, trimethoprim-sulfamethoxazole, troleandomycin, trovafloxacin, or a combination thereof. In some embodiments, the antibacterial agent is selected from mupirocin, gentamycin, tobramycin, or combinations thereof. In one embodiment, the antibacterial agent includes an aminoglycoside. In one embodiment, the antibacterial agent is selected from tetracycline, mupirocin, doxycycline, nitrofurantoin, colistimethate, clindamycin, gentamycin, or combinations thereof.

In various embodiments, the topical composition may comprise an administration dosage of antibacterial agent of up to 2500 mg antibacterial. For example, antibacterial agent may be present in an amount between approximately 10 mg and approximately 2500 mg, such as between approximately 10 and approximately 2000 mg, between approximately 20 mg and approximately 100 mg, between approximately 50 mg and approximately 120, between approximately 80 mg and approximately 120 mg, between approximately 100 mg and approximately 150 mg, or between approximately 120 mg and approximately 170 mg, between approximately 150 mg and approximately 200 mg, between approximately 180 mg and approximately 220 mg, between approximately 200 mg and approximately 250 mg, between approximately 250 mg and approximately 280 mg, between approximately 280 mg and approximately 300 mg, between approximately 300 mg and approximately 360 mg, between approximately 360 mg and approximately 440 mg, between approximately 440 mg and approximately 500 mg, between approximately 500 mg and approximately 600 mg, between approximately 600 mg and approximately 800 mg, between approximately 800 mg and approximately 1000 mg, between approximately 1000 mg and approximately 1200 mg, between approximately 1200 mg and approximately 1400 mg, between approximately 1400 mg and approximately 1600 mg, between approximately 1600 mg and approximately 1800 mg, between approximately 1800 mg and approximately 2000 mg, between approximately 2000 mg and approximately 2500 mg, between approximately 100 mg and approximately 500 mg, between approximately 250 mg and approximately 750 mg, between approximately 500 mg and approximately 1000 mg, between approximately 1000 mg and approximately 1500 mg, or between approximately 750 mg and approximately 1500 mg.

In various embodiments, the one or more anti-infective agents comprises an antibacterial agent selected from one or more antibacterials comprising Vancomycin, ciprofloxacin, levofloxacin, azithromycin, clindamycin, doxycycline, mupirocin, ceftriaxone, colistimethate, tobramycin, cefepime, gentamicin, streptomycin, sulfamethoxazole/trimethoprim, tetracycline, nitrofurantoin.

In one embodiment, the topical composition may include commercially available vancomycin, such as Vancomycin Hydrochloride for Injection, USP, which is a lyophilized powder for preparing intravenous (IV) infusions. The powder may be provided in vials (e.g., bottles) containing the equivalent of 500 mg, 1 g, 5 grams, 10 grams vancomycin base. Other formats may be used, for example Vancomycin Hydrochloride USP powder for oral solution, equivalent to 3.75 g, 7.5 g or 15 g vancomycin, and diluent, which may be a flavored, e.g., grape-flavored, diluent for reconstitution; Vancomycin Intravenous Solution, e.g., vancomycin hydrochloride 5 mg/mL, sodium chloride 9 mg/mL; or Vancomycin Capsules; or bulk powder. In various embodiments, vancomycin may be present in an amount between approximately 1 mg and approximately 100 mg, such as between approximately 5 mg and approximately 50 mg or between approximately 15 mg and approximately 40 mg.

In some embodiments, the topical composition may include commercially available ciprofloxacin, such Ciprofloxacin Hydrochloride Solution/Drops; Ciprofloxacin Hydrochloride Tablets; Ciprofloxacin Tablets, e.g., 500 mg or 100 mg; Ciprofloxacin Hydrochloride Suspension; Ciprofloxacin Injection, USP, e.g., Ciprofloxacin Injection, USP, 20 mL, 200 mg, 1% and 40 mL or 400 mg, 1%, for intravenous injection and infusion, Premix 100 mL in 5% Dextrose, 200 mg, 0.2% and 200 mL in 5% Dextrose or 400 mg, 0.2%, for intravenous infusion; or bulk powder. In various embodiments, ciprofloxacin may be present in an amount between approximately 10 mg and approximately 1500 mg, such as between approximately 200 mg and approximately 1000 mg, approximately 100 mg, approximately 200 mg, approximately 300 mg, approximately 400 mg, or approximately 500 mg.

In certain embodiments, the topical composition may include commercially available levofloxacin, such as Levofloxacin Injection, which may be supplied in single-use vials containing a concentrated solution with the equivalent of 500 mg of levofloxacin USP in 20 mL vials and 750 mg of levofloxacin USP in 30 mL vials; Levofloxacin Solution/Drops; Levofloxacin Tablet; or bulk powder. In various embodiments, levofloxacin may be present in an amount between approximately 10 mg and approximately 1500 mg, such as between approximately 200 mg and approximately 1000 mg or between approximately 400 mg and approximately 750 mg, approximately 250 mg, approximately 500 mg, or approximately 750 mg.

In some embodiments, the topical composition may include commercially available azithromycin, such as Azithromycin for Injection USP, which may be supplied in lyophilized form under a vacuum in a 10 mL vial equivalent to 500 mg of azithromycin for intravenous administration including sodium hydroxide and 413.6 mg citric acid; Azithromycin for Oral Suspension, USP, which may be supplied for suspension in 100 mg/5 mL or 200 mg/5 mL; Azithromycin Tablets; or bulk powder. In various embodiments, Azithromycin may be present in an amount between approximately 10 mg and approximately 1500 mg, such as between approximately 200 mg and approximately 1000 mg, approximately 100 mg, approximately 200 mg, approximately 300 mg, approximately 400 mg, or approximately 500 mg.

In various embodiments, the topical composition may include commercially available clindamycin, such as Clindamycin Phosphate Cream; Clindamycin Phosphate Gel; Clindamycin Phosphate Suspension; Clindamycin Phosphate Injection Solution; Clindamycin Phosphate for Injection; or bulk powder. In various embodiments, clindamycin may be present in an amount between approximately 10 mg and approximately 1500 mg, such as between approximately 200 mg and approximately 1000 mg or between approximately 400 mg and approximately 700 mg, approximately 100 mg, approximately 200 mg, approximately 300 mg, approximately 400 mg, approximately 500 mg, approximately 600 mg, or approximately 700 mg. The clindamycin may include a bulk powder, colistimethate for injection, solution, ground tablets, or a commercially available ointment or cream, for example. Clindamycin may comprise clindamycin phosphate topical solution, USP 1%.

In one embodiment, the topical composition may include commercially available doxycycline, such as Doxycycline Hyclate tablets; Doxycycline Hyclate Tablets; Doxycycline Hyclate Pellets; Doxycycline for Suspension; Doxycycline Hyclate Powder for Suspension; or bulk powder. In various embodiments, doxycycline may be present in an amount between approximately 10 mg and approximately 500 mg, such as between approximately 25 mg and approximately 250 mg or between approximately 50 mg and approximately 150 mg, approximately 50 mg, approximately, 100 mg, approximately 150 mg, or approximately 200 mg.

In certain embodiments, the topical composition may include commercially available mupirocin, such as Mupirocin Ointment; Mupirocin Cream; or bulk powder. In various embodiments, mupirocin may be present in an amount between approximately 1 mg and approximately 120 mg, such as between approximately 5 mg and approximately 100 mg or between approximately 30 mg and approximately 75 mg, approximately 20 mg, approximately 30 mg, approximately 40 mg, or approximately 50 mg.

In some embodiments, the topical composition may include commercially available cefepime, such as Cefepime Hydrochloride Injection, Powder, for Solution, supplied in 500 mg, 1 g, and 2 g vials; Cefepime Hydrochloride Injection Solution; or bulk powder. In various embodiments, cefepime may be present in an amount between approximately 500 mg and approximately 2500 mg, such as between approximately 1000 mg and approximately 2000 mg or between approximately 500 mg and approximately 1000 mg.

In one embodiment, topical composition may include commercially available streptomycin, such as Streptomycin for Injection USP, which may be supplied in 1 g vials; Streptomycin Injection, Powder, Lyophilized, for Solution; or bulk powder. In various embodiments, streptomycin may be present in an amount between approximately 500 mg and approximately 2500 mg, such as between approximately 1000 mg and approximately 2000 mg or between approximately 500 mg and approximately 1000 mg

In some embodiments, the topical composition may include commercially available sulfamethoxazole/trimethoprim, such as Sulfamethoxazole and Trimethoprim Tablets; Sulfamethoxazole and Trimethoprim Injection; Sulfamethoxazole and Trimethoprim Suspension; or bulk powder.

In various embodiments, topical composition may include tetracycline, which may include a commercially available tetracycline format or bulk powder. In various embodiments, tetracycline may be present in an amount between approximately 500 mg and approximately 2500 mg, such as between approximately 1000 mg and approximately 2000 mg or between approximately 1250 mg and approximately 1750 mg, approximately 500 mg, approximately 1000 mg, or approximately 1500 mg. In various embodiments, the tetracycline comprises tetracycline bulk powder, injection solution, cream, ointment, or ground tablets.

In some embodiments, topical composition may include nitrofurantoin, which may include a commercially available nitrofurantoin format or bulk powder. In various embodiments, nitrofurantoin may be present in an amount between approximately 10 mg and approximately 250 mg, such as between approximately 25 mg and approximately 150 mg or between approximately 50 mg and approximately 100 mg. The nitrofurantoin may include a bulk powder, solution, ground tablets, or a commercially available nitrofurantoin ointment or cream, for example.

In various embodiments, the topical composition comprises an antifungal agent, alone or in combination with one or more antibacterial agents, wherein the an antifungal agent includes one or more antifungals selected from one or more categories of antifungal agents including azoles (imidazoles), antimetabolites, allylamines, morpholine, glucan synthesis inhibitors (echinocandins), polyenes, benoxaaborale; other antifungal/onychomycosis agents, and new classes of antifungal/onychomycosis agents. For example, the anti-fungal agent may comprise one or more antifungals selected from abafungin, albaconazole, amorolfin, amphotericin b, anidulafungin, bifonazole, butenafine, butoconazole, candicidin, caspofungin, ciclopirox, clotrimazole, econazole, fenticonazole, filipin, fluconazole, flucytosine, griseofulvin, haloprogin, hamycin, isavuconazole, isoconazole, itraconazole, ketoconazole, micafungin, miconazole, naftifine, natamycin, nystatin, omoconazole, oxiconazole, polygodial, posaconazole, ravuconazole, rimocidin, sertaconazole, sulconazole, terbinafine, terconazole, tioconazole, tolnaftate, undecylenic acid, voriconazole, or a combination thereof. In some embodiments, the antibacterial agent is selected from one or more azoles. In one embodiment, the antifungal agent is selected from itraconazole, voriconazole, or combination thereof.

In various embodiments, the topical composition may comprise an administration dosage of antifungal agent of up to 2500 mg antibacterial. For example, antibacterial agent may be present in an amount between approximately 10 mg and approximately 2500 mg, such as between approximately 10 and approximately 2000 mg, between approximately 20 mg and approximately 100 mg, between approximately 50 mg and approximately 120, between approximately 80 mg and approximately 120 mg, between approximately 100 mg and approximately 150 mg, or between approximately 120 mg and approximately 170 mg, between approximately 150 mg and approximately 200 mg, between approximately 180 mg and approximately 220 mg, between approximately 200 mg and approximately 250 mg, between approximately 250 mg and approximately 280 mg, between approximately 280 mg and approximately 300 mg, between approximately 300 mg and approximately 360 mg, between approximately 360 mg and approximately 440 mg, between approximately 440 mg and approximately 500 mg, between approximately 500 mg and approximately 600 mg, between approximately 600 mg and approximately 800 mg, between approximately 800 mg and approximately 1000 mg, between approximately 1000 mg and approximately 1200 mg, between approximately 1200 mg and approximately 1400 mg, between approximately 1400 mg and approximately 1600 mg, between approximately 1600 mg and approximately 1800 mg, between approximately 1800 mg and approximately 2000 mg, between approximately 2000 mg and approximately 2500 mg, between approximately 100 mg and approximately 500 mg, between approximately 250 mg and approximately 750 mg, between approximately 500 mg and approximately 1000 mg, between approximately 1000 mg and approximately 1500 mg, or between approximately 750 mg and approximately 1500 mg.

In various embodiments, the one or more anti-infective agents comprises an antifungal agent selected from one or more antifungals comprising fluconazole, itraconazole, voriconazole, amphotericin, nystatin, clotrimazole, econazole, or ketoconazole. In some embodiments, the antifungal agent is selected from one or more antifungals comprising itraconazole, voriconazole, clotrimazole, or combination thereof.

In some embodiments, the topical composition may include commercially available fluconazole, such as Fluconazole in Dextrose Inject, Solution; Fluconazole in Sodium Chloride Injection, Solution; Fluconazole Injection; Fluconazole Powder, for Suspension; Fluconazole Tablets; or bulk powder. In various embodiments, fluconazole may be present in an amount between approximately 10 mg and approximately 250 mg, such as between approximately 25 mg and approximately 150 mg or between approximately 50 mg and approximately 100 mg, approximately 50 mg, approximately 100 mg, approximately 150 mg, or approximately 200 mg.

In some embodiments, topical composition may include commercially available itraconazole, such as Itraconazole Capsule; Itraconazole Injection Solution; or bulk powder. In various embodiments, itraconazole may be present in an amount between approximately 5 mg and approximately 150 mg, such as between approximately 20 mg and approximately 150 mg or between approximately 25 mg and approximately 75 mg, approximately 50 mg, or approximately 100 mg.

In various embodiments, the topical composition may include commercially available voriconazole, such as Voriconazole Injection, Powder, Lyophilized, for Solution; Voriconazole Injection, Powder, for Solution; Voriconazole Powder; Voriconazole Tablet; or bulk powder. In various embodiments, voriconazole may be present in an amount between approximately 10 mg and approximately 750 mg, such as between approximately 50 mg and approximately 500 mg or between approximately 100 mg and approximately 300 mg, approximately 100 mg, approximately 150 mg, approximately 200 mg, or approximately 250 mg.

In some embodiments, the topical composition may include commercially available amphotericin, such as Amphotericin B injection, Lipid Complex; Amphotericin B Injection, Powder, Lyophilized, for Solution; or bulk powder. In various embodiments, amphotericin may be present in an amount between approximately 1 mg and approximately 120 mg, such as between approximately 5 mg and approximately 100 mg or between approximately 30 mg and approximately 75 mg, approximately 20 mg, approximately 30 mg, approximately 40 mg, or approximately 50 mg.

In various embodiments, the topical composition may include commercially available nystatin, such as Nystatin Cream; Nystatin Ointment; Nystatin Powder (Topical); or bulk powder.

In some embodiments, topical composition may include commercially available clotrimazole, such as Clotrimazole Cream; Clotrimazole Lotion; Clotrimazole Liquid; Clotrimazole Solution; or bulk powder. In various embodiments, clotrimazole may be present in an amount between approximately 5 mg and approximately 150 mg, such as between approximately 20 mg and approximately 150 mg or between approximately 25 mg and approximately 75 mg, approximately 20 mg, approximately 30 mg, approximately 40 mg, or approximately 50 mg.

In some embodiments, the topical composition may include commercially available econazole, such as Econazole Nitrate Cream; Econazole Nitrate Foam; or bulk powder.

In certain embodiments, the topical composition may include commercially available ketoconazole, such as Ketoconazole Foam; Ketoconazole Cream; Ketoconazole Suspension; Ketoconazole Tablet; Ketoconazole Suspension Shampoo; or bulk powder. In various embodiments, ketoconazole may be present in an amount between approximately 10 mg and approximately 750 mg, such as between approximately 50 mg and approximately 500 mg or between approximately 100 mg and approximately 300 mg, approximately 100 mg, approximately 150 mg, approximately 200 mg, or approximately 250 mg.

As introduced above, in various embodiments, the antibacterial agent or one or more antibacterials thereof, the antifungal agent or one or more antifungals thereof, or combination thereof comprises a commercially available antibacterial or antifungal for injection.

In one example, the topical composition comprises a commercially available tobramycin as tobramycin for injection. Tobramycin for injection is commercially available in various strengths and volumes. For example, tobramycin for injection is currently available in as Tobramycin Injection, USP, 40 mg/1 mL (30 mL), 1.2 g multi-dose vials, 80 mg/2 mL vials, 240 mg/6 mL, 20 mg or 80 mg in 20 mg/2 mL vials. Tobramycin for injection may be provided as powder or solution and may include tobramycin sulfate. In various embodiments, tobramycin may be present in an amount between approximately 5 mg and approximately 300 mg, such as between approximately 20 mg and approximately 250 mg, approximately 25 mg and approximately 200 mg, approximately 50 mg and 100 mg, approximately 20 mg, approximately 40 mg, approximately 60 mg, approximately 80 mg, approximately 100 mg, approximately 120 mg, approximately 160 mg, or approximately 220 mg.

In any of the above or another example, the topical composition comprises a commercially available voriconazole as voriconazole for injection. Voriconazole for injection is commercially available in various strengths and volumes. For example, voriconazole for injection is currently available as Voriconazole for Injection supplied in a single dose vials as a sterile lyophilized powder equivalent to 200 mg voriconazole and 3200 mg sulfobutyl ether beta-cyclodextrin sodium (SBECD) and single doses vial as a sterile lyophilized white to off white cake or powder equivalent to 200 mg voriconazole and 3200 mg hydroxypropyl β-cyclodextrin (HPβCD). In various embodiments, voriconazole may be present in an amount between approximately 5 mg and approximately 400 mg, such as between approximately 20 mg and approximately 350 mg, approximately 50 mg and approximately 250 mg, approximately 100 mg and 200 mg, approximately 20 mg, approximately 50 mg, approximately 75 mg, approximately 100 mg, approximately 150 mg, approximately 180 mg, approximately 200 mg, or approximately 220 mg.

In any of the above or another example, the topical composition comprises commercially available colistimethate as colistimethate for injection. Colistimethate for injection is commercially available in various strengths and volumes. For example, colistimethate for injection, USP is currently available in vials containing colistimethate sodium equivalent to 150 mg colistin base activity per vial, Colistimethate for Injection, USP, 150 mg. In various embodiments, colistimethate may be present in an amount between approximately 10 mg and approximately 1000 mg, such as between approximately 100 mg and approximately 750 mg or between approximately 200 mg and approximately 500 mg, approximately 200 mg, approximately 300 mg, approximately 400 mg, or approximately 500 mg.

In any of the above or another example, the topical composition comprises commercially available ceftriaxone as ceftriaxone for injection. Ceftriaxone for injection is commercially available in various strengths and volumes. For example, Ceftriaxone for injection is currently available as Ceftriaxone for Injection, USP, 500 mg, 250 mg, 2 g, and 1 g. Ceftriaxone Injection, USP, Solution is also available premixed as a frozen, iso-osmotic, sterile, nonpyrogenic solution of ceftriaxone sodium in 1 g equivalent of ceftriaxone, iso-osmotic with approximately 1.9 g Dextrose Hydrous, USP, and 2 gm equivalent of ceftriaxone, iso-osmotic with approximately 1.2 g Dextrose Hydrous, USP. In various embodiments, ceftriaxone may be present in an amount between approximately 10 mg and approximately 1000 mg, such as between approximately 100 mg and approximately 750 mg or between approximately 200 mg and approximately 500 mg, approximately 200 mg, approximately 300 mg, approximately 400 mg, or approximately 500 mg.

In any of the above or another example, the topical composition comprises commercially available levofloxacin as levofloxacin for injection. Levofloxacin for injection is commercially available in various strengths and volumes. For example, levofloxacin for injection is currently available in 500 mg/20 mL strength, 20 mL volume single use container, and in 250 mg/50 mL strength, 50 mL, 100 mL, and 150 mL single-use containers. In various embodiments, levofloxacin may be present in an amount between approximately 500 mg and approximately 2500 mg, such as between approximately 1000 mg and approximately 2000 mg or between approximately 500 mg and approximately 1000 mg, approximately 250 mg, approximately 500 mg, or approximately 750 mg.

In any of the above or another example, the topical composition comprises commercially available linezolid as linezolid for injection. Linezolid for injection may be supplied as a ready-to-use sterile isotonic solution for intravenous infusion. For example, each container may contain 600 mg of linezolid in 300 mL of a clear, colorless to slightly yellow aqueous solution. Inactive ingredients may include: citric acid anhydrous USP 1.92 mg/mL, sodium chloride USP 9 mg/mL, sodium hydroxide NF 0.76 mg/mL, and water for injection USP. Sodium hydroxide NF and/or hydrochloric acid NF are typically used to adjust the pH. The sodium (Na+) content may be about 3.98 mg/mL (52 mEq/300-mL container). Zyvox for injection is supplied as a ready-to-use sterile isotonic solution for intravenous infusion. Each mL contains 2 mg of linezolid. Inactive ingredients are sodium citrate, citric acid, and dextrose in an aqueous vehicle for intravenous administration. The sodium (Na+) content is about 0.38 mg/mL (5 mEq per 300-mL bag; 3.3 mEq per 200-mL bag; and 1.7 mEq per 100-mL bag).

In these or other embodiments, the antibacterial agent or one or more antibacterials thereof, the antifungal agent or one or more antifungals thereof, or combination thereof comprises bulk powder, ground commercial oral tablets, a commercial oral, nasal, or injection solution or suspension, or commercially available topical cream, ointment, foam, lotion, or gel.

For example, the topical composition may include commercially available tobramycin in bulk powder, nasal solution, drops, or powder, ophthalmic suspension, solution, or drops, nebulizer formulas, or combination thereof. In this or another example, the topical composition may include voriconazole in a commercially available in bulk powder, oral suspension or powder for oral suspension, ground tablet, or combination thereof. In any of the above or another example, the topical composition may include linezolid in a commercially available bulk powder format, granules for oral suspension, oral solution, injection dosage, ground oral tablet, or combination thereof. In any of the above or another example, the topical composition may include mupirocin in a commercially available in cream, ointment, lotion, ground oral tablet, bulk powder, or combination thereof. In any of the above or another example, the topical composition may include itraconazole in a commercially available bulk powder, capsule, pellet, or combination thereof. In various embodiments, one or more of the anti-infective agents may be provided in a compounded tablet comprising bulk powder, ground oral tablets, or powder for suspension or solution.

As introduced above, the topical composition may include a diluent comprising one or more diluents. The diluent may be liquid, semi-liquid, or solid. For example, the diluent may include an aqueous, organic, or inorganic solution, cream, gel, ointment, lotion, emulsion, or paste. In some embodiments, the diluent includes a carrier or vehicle composition such as a base cream, ointment, gel, lotion, foam, solution, suspension. The diluent may include lecithin, phospholipids, glycols, paraffin, fatty acids, carbopols, alcohols, lanolin, for example. In some embodiments, the diluent comprises an aqueous solution such as a saline solution. For example, the topical composition may comprise a diluent comprising sodium hydroxide solution, which may be a sterile solution, an alcohol, water, e.g., purified water, water for irrigation, water for injection, or a sterile water. In one embodiment, the diluent comprises a sodium chloride 0.09% solution (sterile). Diluent may be added to a desired volume for irrigation or submersion of the nail or skin area to be treated, which may include surrounding areas, such as between approximately 0.5 L and approximately 4 L, such as between approximately 1 L and approximately 3 L or approximately 1.5 L and approximately 2.5 L.

In various embodiments, the topical composition comprises Diluent dimethyl sulfoxide (DMSO). In some embodiments, a DMSO may be used as a diluent. A commercially available 45.5% DMSO solution, for example, may be used. Other percent concentrations of DMSO may be used, for example DMSO may be present in an amount between approximately 10% and approximately 50%, such as between approximately 15% and approximately 50%, between approximately 20% and approximately 50%, between approximately 25% and approximately 50%, between approximately 30% and approximately 50%, between approximately 35% and approximately 50%, between approximately 40% and approximately 50%, between approximately 20% and approximately 40%, or between approximately 20% and approximately 30%. As described above, DMSO may be applied to nails in a topical nail composition prior to administration of irrigation or footbath formulations to pretreat the nails.

In various embodiments, the topical composition comprises one or more anti-infective agents, as introduced above, and a nonsteroidal anti-inflammatory drug (NSAID) agent. In further embodiments, the topical composition may further include urea, as also introduced above. According to various embodiments, the NSAID agent may include one or more NSAIDS selected from oxicams, such as meloxicam and piroxicam; salicylic acid derivatives, such as aspirin, diflunisal, salsalate, and trilisate; propionic acids, such as flurbiprofen, ibuprofen, ketoprofen, naproxen, or oxaprozin; acetic acids, such as diclofenac, etodolac, indomethacin, ketorolac, nabumetone, sulindac, and tolmetin; fenamates, such as meclofenamate; and/or COX-2 inhibitors, such as celecoxib, rofecoxib, and valdecoxib. All or a portion of the NSAID agent may be obtained from bulk powder. However, in some embodiments, all or a portion of the NSAID agent may be obtained from ground oral tablets, commercial solutions or suspensions, commercial NSAID topical creams such as creams, ointments, lotions, foams, solutions, gels, or combination thereof. For example, in one formula, the NSAID agent comprises the NSAID diclofenac obtained from a commercial diclofenac solution. As described above, NSAID may be applied to nails in a topical nail composition prior to administration of irrigation or footbath formulations to pretreat the nails. In some embodiments, the topical nail composition including one or more NSAIDs may include DMSO.

In various embodiments, the topical composition comprises one or more anti-infective agents and one or more keratolitic agents selected form urea, salicylic acid, papain, or combinations thereof. For example, the topical composition may comprise one or more anti-infective agents and urea. In various embodiments, a topical composition dosage may comprise urea in an amount between approximately 100 mg and approximately 2000 mg, such as between approximately 500 mg and 1500 mg or between approximately 750 mg and 1250 mg, approximately 1000 mg, or approximately 1500 mg.

### Example Topical Compositions

### Example 10

In one embodiment, a topical composition includes doxycycline, tobramycin, mupirocin, and itraconazole. The topical composition may be formulated for administration by footbath or irrigation by combining with a diluent. The doxycycline may be present in an amount between approximately 100 mg and approximately 1000 mg, such as between approximately 300 mg and approximately 900 mg or between approximately 550 mg and approximately 750 mg. The tobramycin may be present in an amount between approximately 100 mg and approximately 1000 mg, such as between approximately 300 mg and approximately 900 mg or between approximately 550 mg and approximately 750 mg. The mupirocin may be present in an amount between approximately 100 mg and approximately 1000 mg, such as between approximately 200 mg and approximately 800 mg or between approximately 350 mg and approximately 550 mg. The itraconazole may be present in an amount between approximately 50 mg and approximately 600 mg, such as between approximately 150 mg and approximately 400 mg or between approximately 200 mg and approximately 300 mg.

A diluent may be combined with the doxycycline, tobramycin, mupirocin, and itraconazole. The diluent may include any suitable diluent, such as those described herein. Example diluents may include water, sodium hydroxide, sodium chloride, saline, sodium hypochlorite, dakin's solution, or combination thereof. The amount of diluent combined may be between approximately 0.5 L and approximately 4 L, such as between approximately 1 L and approximately 3 L or approximately 1.5 L and approximately 2.5 L.

In one embodiment, one or more of doxycycline, tobramycin, mupirocin, or itraconazole may be provided in an ointment for addition with the diluent. In one such example, the doxycycline, tobramycin, mupirocin, and itraconazole may be provided in an ointment comprising, by weight, approximately 2.5% doxycycline, approximately 2.5% tobramycin, approximately 1.855% mupirocin, and approximately 1% itraconazole. Such an ointment may be combined with the diluent to formulate a composition for footbath or irrigation administration.

In one embodiment of Example 10, the topical composition comprises approximately 625 mg doxycycline, approximately 615 mg tobramycin, approximately 464 mg mupirocin, and approximately 250 mg itraconazole. A method of formulating the administration composition using the above ointment formulation may include combining 25 g of the ointment and the diluent. In one example, combining may include adding the diluent to a mixing container, adding 625 mg doxycycline, approximately 615 mg tobramycin, approximately 464 mg mupirocin, and approximately 250 mg itraconazole, e.g., the contents of 1 syringe (25 g), shaking, adding the resultant solution to bath, and placing the affected area(s) into the footbath for 10 minutes. This may be performed up to twice daily or as otherwise needed.

In various embodiments, the topical composition comprising doxycycline, tobramycin, mupirocin, or itraconazole may also include urea. The urea may be present in an amount between approximately 100 mg and approximately 2000 mg, such as between approximately 500 mg and 1500 mg or between approximately 750 mg and 1250 mg. In a further embodiment of the example above, approximately 1000 mg of urea may be combined with the 25 g of the ointment and the diluent. The urea may be a bulk urea powder, ointment, or cream.

In some embodiments, a method of treating a nail infection may include applying a topical nail composition described herein to nails, e.g., a thin layer, prior to a footbath or irrigation administration described herein. The topical nail composition may comprise an NSAID and DMSO. For example, diclofenac and DMSO comprising approximately 1.5% diclofenac and approximately 45.5% DMSO or other formulations described herein may be used. As noted above, the topical composition, with or without urea, may be part of a combination therapy that includes application of one or more additional topical compositions and/or one or more topical nail compositions, or nail lacquers, disclosed herein, which may be applied up to 4 times daily or between footbath or irrigation administration, e.g., once or twice a day.

### Example 11

Another example topical composition may include a first topical composition comprising an antibacterial agent comprising tetracycline. The topical composition may be formulated for administration by combining a diluent. The first topical composition may further include or may be administered as combination treatment with a second topical composition comprising an antibacterial agent comprising mupirocin, an antifungal agent comprising itraconazole. The second topical composition may be formulated by combining the antibacterial agent and the antifungal agent with diluent. In some embodiments, the first and second topical compositions may be formulated into a combined composition or may be used as a standalone treatment or in combination with another topical composition or topical nail composition described herein. The diluent may be any suitable diluent, such as those described herein. Example diluents include water, sodium hydroxide, sodium chloride, saline, sodium hypochlorite, dakin's solution, or combination thereof. The amount of diluent may be between approximately 0.5 L and approximately 4 L, such as between approximately 1 L and approximately 3 L or approximately 1.5 L and approximately 2.5 L. The topical composition may be administered in a bath or irrigation to an affected area, e.g., foot, hand, nails, once daily or as otherwise directed, such as two or more times a day.

In various embodiments, the tetracycline may be present in an amount between approximately 500 mg and approximately 2500 mg, such as between approximately 1000 mg and approximately 2000 mg or between approximately 1250 mg and approximately 1750 mg. In various embodiments, the tetracycline comprises tetracycline bulk powder, injection solution, cream, ointment, or ground tablets. The mupirocin may be present in an amount between approximately 1 mg and approximately 100 mg, such as between approximately 5 mg and approximately 50 mg or between approximately 10 mg and approximately 30 mg. The mupirocin may include a bulk powder, ground tablets, or a commercially available mupirocin ointment or cream, for example. The itraconazole may be present in an amount between approximately 5 mg and approximately 150 mg, such as between approximately 20 mg and approximately 150 mg or between approximately 25 mg and approximately 75 mg. The itraconazole may be a bulk powder, capsule, ground tablet, or other commercially available itraconazole format.

In various embodiments, the first topical composition comprising tetracycline may also include urea. The second topical composition comprising mupirocin and itraconazole may comprise urea. In embodiments wherein the topical composition comprises tetracycline, mupirocin, and itraconazole, the topical composition may similarly comprise urea. The urea may be present in an amount between approximately 100 mg and approximately 2000 mg, such as between approximately 500 mg and 1500 mg or between approximately 750 mg and 1250 mg. In a further embodiment of the example above, approximately 1000 mg of urea may be combined with the diluent and active agents. As noted above, the topical composition may include separate topical composition administrations, in which case the urea may be added to the administration of tetracycline and diluent and/or the administration of mupirocin, itraconazole and diluent. The urea may be a bulk urea powder, ointment, or cream.

As introduced above, a topical compositions may be components of a combination therapy including a topical nail composition comprising an NSAID agent and DMSO, e.g., comprising approximately 1.5% diclofenac and approximately 45.5% DMSO. The NSAID agent and DMSO may be applied to nails in a thin film prior to administration of the topical composition in the footbath or irrigation. As also introduced above, the first and second topical compositions, with or without urea, may comprise a combination therapy or may be part of a combination therapy that includes application of one or more additional topical compositions and/or one or more topical nail compositions, or nail lacquers, disclosed herein. The combination therapy may include sequential or ordered administrations of the member compositions. For example, the first topical composition may be administered once or twice daily and the second topical composition may be administered once or twice daily. Other orders and sequences may also be used. In some embodiments, a topical nail composition comprising DMSO and NSAID may be applied to nails prior to administration of the first and/or second topical composition. In certain embodiments, one or more topical nail compositions may be administered in addition to the first and/or second topical composition, e.g., between, preceding, or after, or may replace one or more administrations of the first and/or second topical compositions.

In one example, the first topical composition includes approximately 1500 mg tetracycline. The composition may be formulated for administration by mixing with a diluent to formulate a solution. The solution may be added to a bath and agitated. The affected area(s) may be bathed for approximately 10 minutes, shorter or longer times may be used. The composition may be administered once or twice daily. The first topical composition may further include or may be administered as combination treatment, as disclosed herein, with a second topical composition comprising approximately 20 mg mupirocin and approximately 50 mg itraconazole prepared and administered as above. For example, approximately 1500 mg tetracycline may be administered once or twice daily and approximately 20 mg mupirocin and approximately 50 mg itraconazole may be administered once or twice daily in a combined administration or in one or more separate administration as disclosed herein.

### Example 12

Another example topical composition may include a first topical composition comprising an antibacterial agent comprising doxycycline and mupirocin and an antifungal agent comprising clotrimazole. The first topical composition may be formulated for administration by combining with a diluent. The first topical composition may further include or may be administered in a combination treatment with a second topical composition comprising an antibacterial agent comprising nitrofurantoin. The second topical composition may be formulated for administration by combining with a diluent. In some embodiments, the first and second topical compositions may be formulated into a combined composition or may be used as a standalone treatment or in combination with another topical composition or topical nail composition described herein. The diluent may be any suitable diluent, such as those described herein. Example diluents include water, sodium hydroxide, sodium chloride, saline, sodium hypochlorite, dakin's solution, or combination thereof. The amount of diluent may be between approximately 0.5 L and approximately 4 L, such as between approximately 1 L and approximately 3 L or approximately 1.5 L and approximately 2.5 L. The topical composition may be administered in a bath or irrigation to an affected area, e.g., foot, hand, nails, once daily or as otherwise directed, such as two or more times a day.

In various embodiments, the doxycycline may be present in an amount between approximately 10 mg and approximately 500 mg, such as between approximately 25 mg and approximately 250 mg or between approximately 50 mg and approximately 150 mg. In various embodiments, the doxycycline comprises doxycycline bulk powder, injection solution, cream, ointment, or ground tablets.

The mupirocin may be present in an amount between approximately 1 mg and approximately 100 mg, such as between approximately 5 mg and approximately 50 mg or between approximately 15 mg and approximately 40 mg. The mupirocin may include a bulk powder, ground tablets, or a commercially available mupirocin ointment or cream, for example. The clotrimazole may be present in an amount between approximately 5 mg and approximately 150 mg, such as between approximately 20 mg and approximately 150 mg or between approximately 25 mg and approximately 75 mg. The clotrimazole may be a bulk powder, capsule, powder for injection, ground tablet, or other commercially available clotrimazole format. The nitrofurantoin may be present in an amount between approximately 10 mg and approximately 250 mg, such as between approximately 25 mg and approximately 150 mg or between approximately 50 mg and approximately 100 mg. The nitrofurantoin may include a bulk powder, solution, ground tablets, or a commercially available nitrofurantoin ointment or cream, for example.

In various embodiments, the first topical composition comprising doxycycline, mupirocin, and clotrimazole may also include urea. The second topical composition comprising nitrofurantoin may also include urea. In embodiments comprising a single topical composition comprising doxycycline, mupirocin, clotrimazole, and nitrofurantoin, the topical composition may similarly include urea. The urea may be present in an amount between approximately 100 mg and approximately 2000 mg, such as between approximately 500 mg and 1500 mg or between approximately 750 mg and 1250 mg. In a further embodiment of the example above, approximately 1000 mg of urea may be combined with the diluent and active agents. As noted above, the topical composition may include separate topical composition administrations, in which case the urea may be added to the administration of doxycycline, mupirocin, and clotrimazole and diluent and/or the administration of nitrofurantoin and diluent. The urea may be a bulk urea powder, ointment, or cream.

As introduced above, the topical compositions, whether combined or in a combination therapy, may further be components of a combination therapy including a topical nail composition comprising an NSAID agent and DMSO, e.g., comprising approximately 1.5% diclofenac and approximately 45.5% DMSO. The NSAID agent and DMSO may be applied to nails in a thin film prior to administration of the topical composition in the footbath or irrigation. As also introduced above, the first topical composition, second topical composition, or a topical composition comprising the ingredients of the first and second topical compositions, with or without urea, may comprise a combination therapy or may be part of a combination therapy that includes application of one or more additional topical compositions and/or one or more topical nail compositions, or nail lacquers, disclosed herein. The combination therapy may include sequential or ordered administrations of the member compositions. For example, the first topical composition may be administered once or twice daily and the second topical composition may be administered once or twice daily. Other orders and sequences may also be used. In some embodiments, a topical nail composition comprising DMSO and NSAID may be applied to nails prior to administration of the first and/or second topical composition. In certain embodiments, one or more topical nail compositions may be administered in addition to the first and/or second topical composition, e.g., between, preceding, or after, or may replace one or more administrations of the first and/or second topical composition.

In one example, the first topical composition includes approximately 100 mg doxycycline, approximately 30 mg mupirocin, and approximately 30 mg clotrimazole. The first topical composition may be formulated for administration by mixing with a diluent to formulate a solution. The solution may be added to a bath and agitated. The affected area(s) may be bathed for approximately 10 minutes, shorter or longer times may be used. The first topical composition may be administered once or twice daily. The first topical composition may further include or may be administered as combination treatment, as disclosed herein, with a second topical composition comprising approximately 75 mg nitrofurantoin prepared and administered as above with respect to the first topical composition. For example, approximately 100 mg doxycycline, approximately 30 mg mupirocin, and approximately 30 mg clotrimazole may be administered once or twice daily and approximately 75 mg nitrofurantoin may be administered once or twice daily in a combined administration or in one or more separate administrations as disclosed herein.

### Example 13

An example topical composition may include a first topical composition comprising an antibacterial agent comprising colistimethate. The first topical composition may be formulated for administration by combining with a diluent. The first topical composition may further include or may be administered in a combination treatment with a second topical composition comprising an antibacterial agent comprising clindamycin. The second topical composition may be formulated for administration by combining with a diluent. The first and/or second topical compositions may further include or be administered in a combination treatment with a third topical composition comprising an antibacterial agent comprising doxycycline and mupirocin and an antifungal agent comprising clotrimazole. In some embodiments, two of or all of the first, second, and third topical compositions may be formulated into a combined composition or may be used as a standalone treatments or in combination with another topical composition or topical nail composition described herein The diluent may be any suitable diluent, such as those described herein. Example diluents include water, sodium hydroxide, sodium chloride, saline, sodium hypochlorite, dakin's solution, or combination thereof. The amount of diluent may be between approximately 0.5 L and approximately 4 L, such as between approximately 1 L and approximately 3 L or approximately 1.5 L and approximately 2.5 L. The topical composition may be administered in a bath or irrigation to an affected area, e.g., foot, hand, nails, once daily or as otherwise directed, such as two or more times a day.

The colistimethate may be present in an amount between approximately 10 mg and approximately 1000 mg, such as between approximately 100 mg and approximately 750 mg or between approximately 200 mg and approximately 500 mg. The colistimethate may include a bulk powder, colistimethate for injection, solution, ground tablets, or a commercially available ointment or cream, for example. The clindamycin may be present in an amount between approximately 10 mg and approximately 1500 mg, such as between approximately 200 mg and approximately 1000 mg or between approximately 400 mg and approximately 700 mg. The clindamycin may include a bulk powder, colistimethate for injection, solution, ground tablets, or a commercially available ointment or cream, for example. Clindamycin may comprise clindamycin phosphate topical solution, USP 1%. In various embodiments, the doxycycline may be present in an amount between approximately 10 mg and approximately 500 mg, such as between approximately 25 mg and approximately 250 mg or between approximately 50 mg and approximately 150 mg. In various embodiments, the doxycycline comprises doxycycline bulk powder, injection solution, cream, ointment, or ground tablets. The mupirocin may be present in an amount between approximately 1 mg and approximately 100 mg, such as between approximately 5 mg and approximately 50 mg or between approximately 15 mg and approximately 40 mg. The mupirocin may include a bulk powder, ground tablets, or a commercially available mupirocin ointment or cream, for example. The clotrimazole may be present in an amount between approximately 5 mg and approximately 150 mg, such as between approximately 20 mg and approximately 150 mg or between approximately 25 mg and approximately 75 mg. The clotrimazole may be a bulk powder, capsule, powder for injection, ground tablet, or other commercially available clotrimazole format.

In various embodiments, the first topical composition comprising colistimethate may also include urea. The second topical composition comprising clindamycin may also include urea. The third topical composition comprising doxycycline, mupirocin, and clotrimazole may also include urea. In embodiments comprising a single topical composition comprising colistimethate, clindamycin, doxycycline, mupirocin, and clotrimazole, the topical composition may similarly include urea. Similarly, embodiments comprising representative combinations of colistimethate, clindamycin, doxycycline, mupirocin, and clotrimazole in two or more topical compositions may also include urea. The urea may be present in an amount between approximately 100 mg and approximately 2000 mg, such as between approximately 500 mg and 1500 mg or between approximately 750 mg and 1250 mg. In a further embodiment of the example above, approximately 1000 mg of urea may be combined with the diluent and active agents. As noted above, the topical composition may include separate topical composition administrations, in which case the urea may be added to the administration of colistimethate and diluent, the administration of clindamycin and diluent, and/or the administration of doxycycline, mupirocin, and clotrimazole and diluent. The urea may be a bulk urea powder, ointment, or cream.

As introduced above, the topical compositions, whether combined or in a combination therapy, may further be components of a combination therapy including a topical nail composition comprising an NSAID agent and DMSO, e.g., comprising approximately 1.5% diclofenac and approximately 45.5% DMSO. The NSAID agent and DMSO may be applied to nails in a thin film prior to administration of the topical composition in the footbath or irrigation. As also introduced above, the first topical composition, second topical composition, or a topical composition comprising the ingredients of the first and second topical compositions, with or without urea, may comprise a combination therapy or may be part of a combination therapy that includes application of one or more additional topical compositions and/or one or more topical nail compositions, or nail lacquers, disclosed herein. The combination therapy may include sequential or ordered administrations of the member compositions. For example, the first topical composition may be administered once or twice daily, the second topical composition may be administered once or twice daily, and the third topical composition may be administered once or twice daily. Other orders and sequences may also be used. In some embodiments, a topical nail composition comprising DMSO and NSAID may be applied to nails prior to administration of the first, second, and/or third topical composition. In certain embodiments, one or more topical nail compositions may be administered in addition to the first, second, and/or third topical composition, e.g., between, preceding, or after, or may replace one or more administrations of the first, second, and/or third topical composition.

In one example, the first topical composition includes approximately 300 mg colistimethate. The first topical composition may be formulated for administration by mixing with a diluent to formulate a solution. The solution may be added to a bath and agitated. The affected area(s) may be bathed for approximately 10 minutes, shorter or longer times may be used. The first topical composition may be administered once or twice daily. The first topical composition may further include or may be administered as combination treatment, as disclosed herein, with a second topical composition and/or a third topical composition. The second topical composition may include approximately 600 mg clindamycin prepared and administered as above with respect to the first topical composition. In one embodiment, the clindamycin is combined from a 60 ml bottle of 1% clindamycin solution, 1% USP. The third topical composition may include approximately 100 mg doxycycline, approximately 30 mg mupirocin, and approximately 30 mg clotrimazole. For example, approximately 300 mg colistimethate may be administered once or twice a day, 600 mg colistimethate may be administered once or twice a day, and/or 100 mg doxycycline, approximately 30 mg mupirocin, and approximately 30 mg clotrimazole may be administered once or twice daily in a combined administration or in one or more separate administrations as disclosed herein.

### Example 14

Another example topical composition may include a first topical composition comprising an antibacterial agent comprising doxycycline and mupirocin and an antifungal agent comprising clotrimazole. The first topical composition may be formulated for administration by combining with a diluent. The first topical composition may further include or may be administered in a combination treatment with a second topical composition comprising an antibacterial agent comprising clindamycin. The second topical composition may be formulated for administration by combining with a diluent. In some embodiments, the first and second topical compositions may be formulated into a combined composition or may be used as a standalone treatment or in combination with another topical composition or topical nail composition described herein. The diluent may be any suitable diluent, such as those described herein. Example diluents include water, sodium hydroxide, sodium chloride, saline, sodium hypochlorite, dakin's solution, or combination thereof. The amount of diluent may be between approximately 0.5 L and approximately 4 L, such as between approximately 1 L and approximately 3 L or approximately 1.5 L and approximately 2.5 L. The topical composition may be administered in a bath or irrigation to an affected area, e.g., foot, hand, nails, once daily or as otherwise directed, such as two or more times a day.

In various embodiments, the doxycycline may be present in an amount between approximately 10 mg and approximately 500 mg, such as between approximately 25 mg and approximately 250 mg, approximately 150 mg to approximately 250 mg, or between approximately 50 mg and approximately 150 mg. In various embodiments, the doxycycline comprises doxycycline bulk powder, injection solution, cream, ointment, or ground tablets. The mupirocin may be present in an amount between approximately 1 mg and approximately 100 mg, such as between approximately 5 mg and approximately 50 mg or between approximately 15 mg and approximately 40 mg. The mupirocin may include a bulk powder, ground tablets, or a commercially available mupirocin ointment or cream, for example.

The clotrimazole may be present in an amount between approximately 5 mg and approximately 150 mg, such as between approximately 20 mg and approximately 150 mg or between approximately 25 mg and approximately 75 mg. The clotrimazole may be a bulk powder, capsule, powder for injection, ground tablet, or other commercially available clotrimazole format.

The clindamycin may be present in an amount between approximately 10 mg and approximately 1500 mg, such as between approximately 200 mg and approximately 1000 mg or between approximately 400 mg and approximately 700 mg. The clindamycin may include a bulk powder, colistimethate for injection, solution, ground tablets, or a commercially available ointment or cream, for example. Clindamycin may comprise clindamycin phosphate topical solution, USP 1%.

In various embodiments, the first topical composition comprising doxycycline, mupirocin, and clotrimazole may also include urea. The second topical composition comprising clindamycin may also include urea. In embodiments comprising a single topical composition comprising doxycycline, mupirocin, clotrimazole, and clindamycin, the topical composition may similarly include urea. The urea may be present in an amount between approximately 100 mg and approximately 2000 mg, such as between approximately 500 mg and 1500 mg or between approximately 750 mg and 1250 mg. In a further embodiment of the example above, approximately 1000 mg of urea may be combined with the diluent and active agents. As noted above, the topical composition may include separate topical composition administrations, in which case the urea may be added to the administration of doxycycline, mupirocin, and clotrimazole and diluent and/or the administration of clindamycin and diluent. The urea may be a bulk urea powder, ointment, or cream.

As introduced above, the topical compositions, whether combined or in a combination therapy, may further be components of a combination therapy including a topical nail composition comprising an NSAID agent and DMSO, e.g., comprising approximately 1.5% diclofenac and approximately 45.5% DMSO. The NSAID agent and DMSO may include a solution for application to nails in a thin film prior to administration of the topical composition in the footbath or irrigation. As also introduced above, the first topical composition, second topical composition, or a topical composition comprising the ingredients of the first and second topical compositions, with or without urea, may comprise a combination therapy or may be part of a combination therapy that includes application of one or more additional topical compositions and/or one or more topical nail compositions, or nail lacquers, disclosed herein. The combination therapy may include sequential or ordered administrations of the member compositions. For example, the first topical composition may be administered once or twice daily and the second topical composition may be administered once or twice daily. Other orders and sequences may also be used. In some embodiments, a topical nail composition comprising DMSO and NSAID may be applied to nails prior to administration of the first and/or second topical composition. In certain embodiments, one or more topical nail compositions may be administered in addition to the first and/or second topical composition, e.g., between, preceding, or after, or may replace one or more administrations of the first and/or second topical compositions.

In one example, the first topical composition includes approximately 100 mg doxycycline, approximately 30 mg mupirocin, and approximately 30 mg clotrimazole. The first topical composition may be formulated for administration by mixing with a diluent to formulate a solution. The solution may be added to a bath and agitated. The affected area(s) may be bathed for approximately 10 minutes, shorter or longer times may be used. The first topical composition may be administered once or twice daily. The first topical composition may further include or may be administered as combination treatment, as disclosed herein, with a second topical composition. The second topical composition may include approximately 600 mg clindamycin prepared and administered as above with respect to the first topical composition. In one embodiment, the clindamycin is combined from a 60 ml bottle of 1% clindamycin solution, 1% USP. In one example, approximately 100 mg doxycycline, approximately 30 mg mupirocin, and approximately 30 mg clotrimazole may be administered once or twice daily and approximately 600 mg clindamycin may be administered once or twice daily in a combined administration or in one or more separate administrations as disclosed herein.

In one example, a topical composition includes approximately 100 mg doxycycline, approximately 30 mg mupirocin, approximately 30 mg clotrimazole, approximately 600 mg clindamycin. The first topical composition may be formulated for administration by mixing with a diluent to formulate a solution. The solution may be added to a bath and agitated. The affected area(s) may be bathed for approximately 10 minutes, shorter or longer times may be used. The first topical composition may be administered once or twice daily.

### Example 15

Another example topical composition may include an antibacterial agent comprising doxycycline and mupirocin and an antifungal agent comprising clotrimazole. The topical composition may be formulated for administration by combining with a diluent. The topical composition may further include or may be administered in a combination treatment with a second topical composition or topical nail composition described herein. The diluent may be any suitable diluent, such as those described herein. Example diluents include water, sodium hydroxide, sodium chloride, saline, sodium hypochlorite, dakin's solution, or combination thereof. The amount of diluent may be between approximately 0.5 L and approximately 4 L, such as between approximately 1 L and approximately 3 L or approximately 1.5 L and approximately 2.5 L. The topical composition may be administered in a bath or irrigation to an affected area, e.g., foot, hand, nails, once daily or as otherwise directed, such as two or more times a day.

In various embodiments, the doxycycline may be present in an amount between approximately 10 mg and approximately 500 mg, such as between approximately 25 mg and approximately 250 mg, approximately 150 mg to approximately 250 mg, or between approximately 50 mg and approximately 150 mg. In various embodiments, the doxycycline comprises doxycycline bulk powder, injection solution, cream, ointment, or ground tablets. The mupirocin may be present in an amount between approximately 1 mg and approximately 120 mg, such as between approximately 5 mg and approximately 100 mg or between approximately 30 mg and approximately 75 mg. The mupirocin may include a bulk powder, ground tablets, or a commercially available mupirocin ointment or cream, for example. The clotrimazole may be present in an amount between approximately 5 mg and approximately 150 mg, such as between approximately 20 mg and approximately 150 mg or between approximately 25 mg and approximately 75 mg. The clotrimazole may be a bulk powder, capsule, powder for injection, ground tablet, or other commercially available clotrimazole format.

In various embodiments, the topical composition comprising doxycycline, mupirocin, and clotrimazole may also include urea. The urea may be present in an amount between approximately 100 mg and approximately 2000 mg, such as between approximately 500 mg and 1500 mg or between approximately 750 mg and 1250 mg. In a further embodiment of the example above, approximately 1000 mg of urea may be combined with the diluent and active agents. The urea may be a bulk urea powder, ointment, or cream.

As introduced above, the topical composition may be a component of a combination therapy including a topical nail composition comprising an NSAID agent and DMSO, e.g., comprising approximately 1.5% diclofenac and approximately 45.5% DMSO. The NSAID agent and DMSO may be applied to nails in a thin film prior to administration of the topical composition in the footbath or irrigation. As also introduced above, the topical composition, with or without urea, may be part of a combination therapy that includes application of one or more additional topical compositions and/or one or more topical nail compositions, or nail lacquers, disclosed herein.

In one example, the topical composition includes approximately 200 mg doxycycline, approximately 60 mg mupirocin, and approximately 60 mg clotrimazole. The topical composition may be formulated for administration by mixing with a diluent to formulate a solution. The solution may be added to a bath and agitated. The affected area(s) may be bathed for approximately 10 minutes, shorter or longer times may be used. The topical composition may be administered once or twice daily. The topical composition may further include or may be administered as combination treatment, as disclosed herein, with one or more additional topical compositions and/or topical nail compositions.

### Example 16

An example topical composition may include a first composition comprising an antibacterial agent comprising colistimethate. The first composition may be formulated for administration by combining with a diluent. The first composition may further include or may be administered in a combination treatment with a second composition comprising an antifungal agent comprising voriconazole. The second composition may be formulated for administration by combining with a diluent. The first and/or second compositions may further include or be administered in a combination treatment with a third composition comprising an antibacterial agent comprising doxycycline and mupirocin. In some embodiments, two of or all of the first, second, and third compositions may be formulated into a combined topical composition or may be used as a standalone treatments or in combination with another topical composition or topical nail composition described herein. For example, the topical composition may comprise the first, second, and third compositions formulated in a single topical composition for administration to a foot or hand in a footbath or via irrigation. The diluent may be any suitable diluent, such as those described herein. Example diluents include water, sodium hydroxide, sodium chloride, saline, sodium hypochlorite, dakin's solution, or combination thereof. The amount of diluent may be between approximately 0.5 L and approximately 4 L, such as between approximately 1 L and approximately 3 L or approximately 1.5 L and approximately 2.5 L. The topical composition may be administered in a bath or irrigation to an affected area, e.g., foot, hand, nails, once daily or as otherwise directed, such as two or more times a day.

In various embodiments, the doxycycline may be present in an amount between approximately 10 mg and approximately 750 mg, such as between approximately 50 mg and approximately 500 mg or between approximately 100 mg and approximately 250 mg. In various embodiments, the doxycycline comprises doxycycline bulk powder, injection solution, cream, ointment, or ground tablets. The mupirocin may be present in an amount between approximately 1 mg and approximately 100 mg, such as between approximately 5 mg and approximately 75 mg or between approximately 10 mg and approximately 50 mg. The mupirocin may include a bulk powder, ground tablets, or a commercially available mupirocin ointment or cream, for example.

The colistimethate may be present in an amount between approximately 10 mg and approximately 1000 mg, such as between approximately 100 mg and approximately 750 mg or between approximately 200 mg and approximately 500 mg. The colistimethate may include a bulk powder, colistimethate for injection, solution, ground tablets, or a commercially available ointment or cream, for example.

The voriconazole may be present in an amount between approximately 10 mg and approximately 750 mg, such as between approximately 50 mg and approximately 500 mg or between approximately 100 mg and approximately 300 mg. The voriconazole may be a bulk powder, capsule, powder for injection, ground tablet, or other commercially available voriconazole format.

In various embodiments, one or more of the first, second, or third compositions may comprise urea. For example, the first composition comprising colistimethate may also include urea. The second composition comprising voriconazole may also include urea. The third composition comprising doxycycline and mupirocin may also include urea. In embodiments comprising a combined topical composition comprising colistimethate, voriconazole, doxycycline, and mupirocin, the topical composition may similarly include urea. Similarly, embodiments comprising representative combinations of colistimethate, voriconazole, doxycycline, and mupirocin in two or more topical compositions may also include urea. The urea may be present in an amount between approximately 100 mg and approximately 2000 mg, such as between approximately 500 mg and 1500 mg or between approximately 750 mg and 1250 mg. In a further embodiment of the example above, approximately 1000 mg of urea may be combined with the diluent and active agents. The urea may be a bulk urea powder, ointment, or cream.

As introduced above, the topical composition may be a component of a combination therapy including a topical nail composition comprising an NSAID agent and DMSO, e.g., comprising approximately 1.5% diclofenac and approximately 45.5% DMSO. The NSAID agent and DMSO may be applied to nails in a thin film prior to administration of the topical composition in the footbath or irrigation. As also introduced above, the first, second, and/or third topical compositions, with or without urea, may comprise a combination therapy or may be part of a combination therapy that includes application of one or more additional topical compositions and/or one or more topical nail compositions, or nail lacquers, disclosed herein. The combination therapy may include sequential or ordered administrations of the member compositions. For example, the first topical composition may be administered once or twice daily, the second topical composition may be administered once or twice daily, and the third topical composition may be administered once or twice daily. Other orders and sequences may also be used. In some embodiments, a topical nail composition comprising DMSO and NSAID may be applied to nails prior to administration of the first, second, and/or third topical composition. In certain embodiments, one or more topical nail compositions may be administered in addition to the first, second, and/or third topical composition, e.g., between, preceding, or after, or may replace one or more administrations of the first, second, and/or third topical composition.

In one example, the topical composition includes approximately 200 mg doxycycline, approximately 30 mg mupirocin, approximately 300 mg clotrimazole, and approximately 200 mg voriconazole. The topical composition may be formulated for administration by mixing with a diluent to formulate a solution. The solution may be added to a bath and agitated. The affected area(s) may be bathed for approximately 10 minutes, shorter or longer times may be used. The topical composition may be administered once or twice daily. The first topical composition may further include or may be administered as combination treatment, as disclosed herein, with one or more additional topical compositions and/or topical nail compositions.

### Example 17

An example topical composition may include an antibacterial agent comprising clindamycin, mupirocin, and doxycycline and an antifungal agent comprising voriconazole. The topical composition may be formulated for administration by combining with a diluent. In another example, a combination treatment comprises a first composition comprising a first topical composition including clindamycin, a second composition comprising a second topical composition comprising voriconazole, and a third composition comprising a third topical composition including mupirocin and doxycycline. Each of the first, second, and third components may be mixed with a diluent prior to administration in a footbath or via irrigation. In some embodiments, two of or all of the first, second, and third compositions may be formulated into a combined topical composition or may be used as a standalone treatments or in combination with another topical composition or topical nail composition described herein. For example, a topical composition may comprise the first, second, and third compositions formulated in a single topical composition for administration to a foot or hand in a footbath or via irrigation.

Example diluents include water, sodium hydroxide, sodium chloride, saline, sodium hypochlorite, dakin's solution, or combination thereof. The amount of diluent may be between approximately 0.5 L and approximately 4 L, such as between approximately 1 L and approximately 3 L or approximately 1.5 L and approximately 2.5 L for each administration. The topical composition may be administered in a bath or irrigation to an affected area, e.g., foot, hand, nails, once daily or as otherwise directed, such as two or more times a day.

In various embodiments, the voriconazole may be present in an amount between approximately 10 mg and approximately 750 mg, such as between approximately 50 mg and approximately 500 mg or between approximately 100 mg and approximately 300 mg. The voriconazole may be a bulk powder, capsule, powder for injection, ground tablet, or other commercially available voriconazole format.

In various embodiments, the clindamycin may be present in an amount between approximately 10 mg and approximately 1500 mg, such as between approximately 200 mg and approximately 1000 mg or between approximately 400 mg and approximately 700 mg. The clindamycin may include a bulk powder, colistimethate for injection, solution, ground tablets, or a commercially available ointment or cream, for example. Clindamycin may comprise clindamycin phosphate topical solution, USP 1%.

In various embodiments, doxycycline may be present in an amount between approximately 10 mg and approximately 750 mg, such as between approximately 50 mg and approximately 500 mg or between approximately 100 mg and approximately 250 mg. In various embodiments, the doxycycline comprises doxycycline bulk powder, injection solution, cream, ointment, or ground tablets. The mupirocin may be present in an amount between approximately 1 mg and approximately 100 mg, such as between approximately 5 mg and approximately 75 mg or between approximately 10 mg and approximately 50 mg. The mupirocin may include a bulk powder, ground tablets, or a commercially available mupirocin ointment or cream, for example.

In various embodiments, one or more of the first, second, or third compositions or topical compositions may comprise urea. For example, the first composition or formulated first topical composition comprising clindamycin may also include urea. The second topical composition or formulated second topical composition comprising voriconazole may also include urea. The third composition or formulated third composition comprising doxycycline and mupirocin may also include urea. In embodiments comprising a single topical composition comprising clindamycin, voriconazole, doxycycline, and mupirocin, the topical composition may similarly include urea. Similarly, embodiments comprising representative combinations of clindamycin, voriconazole, doxycycline, and mupirocin in two or more topical compositions may also include urea. The urea may be present in an amount between approximately 100 mg and approximately 2000 mg, such as between approximately 500 mg and 1500 mg or between approximately 750 mg and 1250 mg. In a further embodiment of the example above, approximately 1000 mg of urea may be combined with the diluent and active agents. The urea may be a bulk urea powder, ointment, or cream.

As introduced above, the topical composition or topical compositions may be a part of a combination therapy including a topical nail composition comprising an NSAID agent and DMSO, e.g., comprising approximately 1.5% diclofenac and approximately 45.5% DMSO. The NSAID agent and DMSO may be applied to nails in a thin film prior to administration of the topical composition in the footbath or irrigation. As also introduced above, the first, second, and/or third topical compositions, with or without urea, may comprise a combination therapy or may be part of a combination therapy that includes application of one or more additional topical compositions and/or one or more topical nail compositions, or nail lacquers, disclosed herein. The combination therapy may include sequential or ordered administrations of the member compositions. For example, the first topical composition may be administered once or twice daily, the second topical composition may be administered once or twice daily, and the third topical composition may be administered once or twice daily. Other orders and sequences may also be used. In some embodiments, a topical nail composition comprising DMSO and NSAID may be applied to nails prior to administration of the first, second, and/or third topical composition. In certain embodiments, one or more topical nail compositions may be administered in addition to the first, second, and/or third topical composition, e.g., between, preceding, or after, or may replace one or more administrations of the first, second, and/or third topical composition.

In one example, the topical composition includes approximately 200 mg doxycycline, approximately 30 mg mupirocin, approximately 600 mg clindamycin, and approximately 200 mg voriconazole. The topical composition may be formulated for administration by mixing with a diluent to formulate a solution. In one example, the first composition includes approximately 600 mg clindamycin, the second composition includes approximately 200 mg voriconazole, and the third composition comprises approximately 200 mg doxycycline and approximately 30 mg mupirocin. Respective topical compositions may be formulated for administration by mixing with a diluent to formulate a solution. In one embodiment, the clindamycin is combined from a 60 ml bottle of 1% clindamycin solution, 1% USP. The solution may be added to a bath and agitated. The affected area(s) may be bathed for approximately 10 minutes, shorter or longer times may be used. The topical compositions may be administered once or twice daily. The topical compositions may further include or may be administered as combination treatment, as disclosed herein, with one or more additional topical compositions and/or topical nail compositions.

It will be appreciated that the various example topical compositions including first and second topical compositions may be rearranged for use with other topical compositions and/or first topical compositions and/or second topical compositions.

### Combination Treatments

Various combination treatments are disclosed above and elsewhere herein. In some embodiments, combination treatments may include two or more topical nail compositions, two or more topical compositions, or one or more topical nail compositions and one or more topical compositions.

In various embodiments, a combination treatment including a topical nail composition and/or a topical composition for administration in a footbath or by irrigation may include a first composition comprising one or more antibacterial agents and a second composition comprising one or more antifungal agents. In some embodiments, the first composition comprises one or more antibacterial agents and one or more antifungal agents. In these or other embodiments, the second composition may include one or more antifungal agents and one or more antibacterial agents. The compositions may be administered sequentially, e.g., back to back, which may be spaced apart throughout a day, e.g., an administration occurring every 6 to 8 or 6 to 12 hours. In some embodiments, a combination treatment may include a treatment with a first composition comprising a topical composition for administration in a footbath or by irrigation and a second composition comprising a topical nail composition. The compositions may be administered alternately or immediately following the other. In one example, the first composition comprises a topical composition for administration in a footbath or by irrigation. The first composition may comprise two separate compositions comprising different active agents as disclosed herein. The second composition may comprise a topical nail composition. The second composition may comprise two separate compositions comprising different active agents as disclosed herein. The selection of the separate compositions for administration may alternate by day or administration occurrence. One or more of the compositions may include urea as disclosed herein. Additionally or alternatively, the combination treatment may include a composition comprising an NSAID and DMSO as disclosed herein for administration to nails.

In various embodiments, a combination treatment includes a combination of compositions disclosed herein. For example, a combination treatment may include a first composition comprising the topical nail composition of Example 1 and one or more second and/or third compositions comprising one or more topical nail compositions and/or topical compositions selected from Examples 2-17 and combinations thereof, such as combinations disclosed herein. In one example, a combination treatment may include a first composition comprising the topical nail composition of Example 2 and one or more second and/or third compositions comprising one or more topical nail compositions and/or topical compositions selected from Examples 1 and 3-17 and combinations thereof, such as combinations disclosed herein. In another example, a combination treatment may include a first composition comprising the topical nail composition of Example 3 and one or more second and/or third compositions comprising one or more topical nail compositions and/or topical compositions selected from Examples 1, 2, and 4-17 and combinations thereof, such as combinations disclosed herein. In a still another, a combination treatment may include a first composition comprising the topical nail composition of Example 4 and one or more second and/or third compositions comprising one or more topical nail compositions and/or topical compositions selected from Examples 1-3 and 5-17 and combinations thereof, such as combinations disclosed herein. In yet another example, a combination treatment may include a first composition comprising the topical nail composition of Example 5 and one or more second and/or third compositions comprising one or more topical nail compositions and/or topical compositions selected from Examples 1-4 and 6-17 and combinations thereof, such as combinations disclosed herein. In still another example, a combination treatment may include a first composition comprising the topical nail composition of Example 6 and one or more second and/or third compositions comprising one or more topical nail compositions and/or topical compositions selected from Examples 1-5 and 7-17 and combinations thereof, such as combinations disclosed herein. In still yet another example, a combination treatment may include a first composition comprising the topical nail composition of Example 7 and one or more second and/or third compositions comprising one or more topical nail compositions and/or topical compositions selected from Examples 1-6 and 8-17 and combinations thereof, such as combinations disclosed herein. In one example, a combination treatment may include a first composition comprising the topical nail composition of Example 8 and one or more second and/or third compositions comprising one or more topical nail compositions and/or topical compositions selected from Examples 1-7 and 9-17 and combinations thereof, such as combinations disclosed herein. In another example, a combination treatment may include a first composition comprising the topical nail composition of Example 9 and one or more second and/or third compositions comprising one or more topical nail compositions and/or topical compositions selected from Examples 1-8 and 10-17 and combinations thereof, such as combinations disclosed herein. In still another example, a combination treatment may include a first composition comprising the topical composition of Example 10 and one or more second and/or third compositions comprising one or more topical nail compositions and/or topical compositions selected from Examples 1-9 and 11-17 and combinations thereof, such as combinations disclosed herein. In yet another example, a combination treatment may include a first composition comprising the topical composition of Example 11, or the first or second topical composition thereof, and one or more second and/or third compositions comprising one or more topical nail compositions and/or topical compositions selected from Examples 1-10 and 12-17 and combinations thereof, such as combinations disclosed herein. In still yet example, a combination treatment may include a first composition comprising the topical composition of Example 12, or the first or second topical composition thereof, and one or more second and/or third compositions comprising one or more topical nail compositions and/or topical compositions selected from Examples 1-11 and 13-17 and combinations thereof, such as combinations disclosed herein. In one example, a combination treatment may include a first composition comprising the topical composition of Example 13, or the first, second, and/or third, topical composition thereof, and one or more second and/or third compositions comprising one or more topical nail compositions and/or topical compositions selected from Examples 1-12 and 14-17 and combinations thereof, such as combinations disclosed herein. In another example, a combination treatment may include a first composition comprising the topical composition of Example 14, or the first or second, and one or more second and/or third compositions comprising one or more topical nail compositions and/or topical compositions selected from Examples 1-13 and 15-17 and combinations thereof, such as combinations disclosed herein. In still another example, a combination treatment may include a first composition comprising the topical composition of Example 15 and one or more second and/or third compositions comprising one or more topical nail compositions and/or topical compositions selected from Examples 1-14, 16, and 17 and combinations thereof, such as combinations disclosed herein. In yet another example, a combination treatment may include a first composition comprising the topical composition of Example 16, or the first, second, and/or third, and one or more second and/or third compositions comprising one or more topical nail compositions and/or topical compositions selected from Examples 1-15 and 17 and combinations thereof, such as combinations disclosed herein. In still yet another example, a combination treatment may include a first composition comprising the topical composition of Example 17, or the first, second, and/or third, and one or more second and/or third compositions comprising one or more topical nail compositions and/or topical compositions selected from Examples 1-16 and combinations thereof, such as combinations disclosed herein.

In some embodiments, a topical bacterial or fungal nail infection treatment includes a combination treatment including any of the topical nail compositions and/or topical compositions for combination administration disclosed above and elsewhere herein. For example topical bacterial or fungal nail infection treatments comprising topical nail compositions for combination administration, e.g., comprising a combination treatment, may also include a first composition comprising a topical nail composition selected from Examples 1-3 and one or more second compositions selected from the topical nail compositions of Examples 4 and 5. For example, a first composition comprising the topical nail composition of Example 1 and a second composition selected from a topical nail composition of Example 4 or Example 5; a first topical comprising the topical nail composition of Example 2 and a second composition selected from a topical nail composition of Example 4 or Example 5; a first composition comprising the topical nail composition of Example 3 and a second composition selected from a topical nail composition of Example 4 or Example 5; a first composition comprising the topical nail composition of Example 4 and a second composition selected from a topical nail composition of Example 1, Example 2, or Example 3; or first composition comprising the topical nail composition of Example 5 and a second composition selected from a topical nail composition of Example 1, Example 2, or Example 3.

Another combination treatment may include a first composition comprising a topical nail composition comprising the topical nail composition of Example 6 and a second composition comprising the topical nail composition of Example 7. Another combination administration may include a first composition comprising the topical nail composition of Example 8 and a second composition comprising the topical nail composition of Example 9. Another combination administration may include a first composition comprising the topical nail composition of Example 6 and a second composition comprising the topical nail composition of Example 8. Another combination administration may include a first composition comprising the topical nail composition of Example 7 and a second composition comprising the topical nail composition of Example 9.

Additional example combination treatments may include (a) a first composition comprising a topical nail composition selected from Examples 1-3 and 8 and one or more second topical nail compositions selected from Examples 4-7 and 9; (b) a first composition comprising a topical nail composition selected from Examples 4, 6, and 9 and one or more second compositions comprising a topical nail composition selected from Examples 1-3, 5, 7, 8, and combinations thereof; (c) a first composition comprising a topical nail composition selected from Examples 1-9 and one or more second compositions selected from a topical composition for administration by irrigation or in a footbath of Examples 10-17 and/or first, second, and/or third topical compositions thereof.

The topical nail compositions for combination treatment may each include one or more anti-infectives, e.g., antibacterial and/or antifungal agents, disclosed herein. One or more of the topical nail compositions of a combination treatment may also include or be administered following administration of a topical nail composition comprising an NSAID and DMSO solution as disclosed herein, e.g., a diclofenac and DMSO solution, such as a solution comprising approximately 1.5% diclofenac and approximately 45.5% DMSO.

As noted above, the topical compositions of Examples 11-17 may comprise combination treatments. Additional example combination treatments may include a first and one or more second (or third) compositions selected from combinations of first, second, and/or third topical compositions of Examples 11-17.

In one example, a combination treatment includes a first composition comprising the first topical composition of Example 11 and one or more second compositions selected from the second topical composition of Example 12, the first topical composition of Example 13, the second topical composition of Example 13, the third topical composition of Example 13, the second topical composition of Example 16, the third topical composition of Example 16, or combination thereof.

In another example, a combination treatment includes a first composition comprising the second topical composition of Example 12 and one or more second compositions selected from the first topical composition of Example 11, the first topical composition of Example 13, the second topical composition of Example 13, the third topical composition of Example 13, the second topical composition of Example 16, the third topical composition of Example 16, or combination thereof.

In another example, a combination treatment includes a first composition comprising the first topical composition of Example 13 and one or more second compositions selected from the first topical composition of Example 11, the second topical composition of Example 12, the second topical composition of Example 13, the third topical composition of Example 13, the second topical composition of Example 16, the third topical composition of Example 16, or combination thereof.

In another example, a combination treatment includes a first composition comprising the second topical composition of Example 13 and one or more second compositions selected from the first topical composition of Example 11, the second topical composition of Example 12, the first topical composition of Example 13, the third topical composition of Example 13, the second topical composition of Example 16, the third topical composition of Example 16, or combination thereof.

In another example, a combination treatment includes a first composition comprising the second topical composition of Example 16 and one or more second compositions selected from the first topical composition of Example 11, the second topical composition of Example 12, the first topical composition of Example 13, the second topical composition of Example, 13 the third topical composition of Example 13, the third topical composition of Example 16, or combination thereof.

The topical compositions for combination treatment may each include one or more anti-infectives, e.g., antibacterial and/or antifungal agents, disclosed herein. One or more of the topical compositions of a combination treatment may also include or be administered following administration of a topical nail composition comprising an NSAID and DMSO solution as disclosed herein, e.g., a diclofenac and DMSO solution, such as a solution comprising approximately 1.5% diclofenac and approximately 45.5% DMSO.

In further examples, the combination treatments above may further comprise one or more additional compositions selected from the topical nail compositions of Examples 1-9 and combinations thereof, such as those disclosed herein.

In various embodiments, combination treatments including topical nail compositions may be sequentially administered, alternating among the topical nail compositions or may be administered in various sequenced or ordered combinations, such as those disclosed herein. For example, a treatment may include a sequence of administrations wherein a first composition comprising a topical nail composition may be administered in a first daily administration and a second composition comprising a topical nail composition, different from the first, may be administered in a second daily administration. In various embodiments, topical compositions of a combination treatment may be sequentially administered, alternating among the topical compositions or may be administered in various sequenced or ordered combinations, such as those disclosed herein. For example, a treatment may include a sequence of administrations wherein a first composition comprising a topical composition may be administered in a first daily administration and a second composition comprising a topical composition, different from the first, may be administered in a second daily administration. In some embodiments, topical compositions and topical nail compositions of a combination treatment may be sequentially administered, alternating among the topical compositions and topical nail compositions or may be administered in various sequenced or ordered combinations, such as those disclosed herein. For example, a treatment may include a sequence of administrations wherein a first composition comprising a topical composition may be administered in a first daily administration and a second composition comprising a topical nail composition may be administered in a second daily administration.

In some embodiments, the first and second compositions may be separately administered once daily or twice daily, for example. In one embodiment, the first composition may be administered twice daily and the second composition once daily. In a further embodiment, the first and second compositions may rotate every other day with respect to the composition that is administered twice and the composition that is administered once.

In further example administration protocols, a first composition may be administered in one or two daily administrations every other day, for a first two or three days, or a first week, for example. The second composition may be administered in one or two daily administrations on the days the first composition is not administered. Other sequences may be used. For example, the first and second compositions may alternate two on, one off, or three on and one off or two off. In some embodiments, a combination treatment includes one or more additional second/third compositions comprising one or more topical nail compositions and/or topical compositions for administration by irrigation or footbath. As noted above, a combination treatment may include an NSAID and DMSO topical nail composition may be administered prior to administration of a first and/or second composition, which may include topical compositions and/or other topical nail compositions.

### Methods of Treating a Bacterial or Fungal Nail Infection

A method of treating a bacterial or fungal nail infection may include topically applying any topical nail composition disclosed herein. For example a topical nail composition may be directly applied to an affected nail surface. In some embodiments, topical administration to the affected nail surface may include layering or coating the nail surface with the topical nail composition. The topical nail composition may be applied directly to the nail surface via a brush, sponge, dropper, syringe, or other applicator. In one example, the topical nail composition may be painted onto the nail surface. The topical nail composition may be administered 1 to 4 times daily or as otherwise directed to treat the nail infection.

According to one embodiment, a method of pretreating a bacterial or fungal nail infection may include applying a solution containing a NSAID and DMSO to a nail before applying the topical nail composition to the nail. The solution containing the NSAID and DMSO may be as disclosed herein. For example, the solution may include one or more NSAIDS selected from oxicams, such as meloxicam and piroxicam; salicylic acid derivatives, such as aspirin, diflunisal, salsalate, and trilisate; propionic acids, such as flurbiprofen, ibuprofen, ketoprofen, naproxen, or oxaprozin; acetic acids, such as diclofenac, etodolac, indomethacin, ketorolac, nabumetone, sulindac, and tolmetin; fenamates, such as meclofenamate; and/or COX-2 inhibitors, such as celecoxib, rofecoxib, and valdecoxib. In one embodiment, the NSAID includes diclofenac. Diclofenac may be present in an amount between approximately 0.05% and approximately 5%, such as between approximately 1% and approximately 3%, between approximately 1% and approximately 2%, or approximately 1.5%. DMSO may be present in an amount between approximately 10% and approximately 50%, such as between approximately 15% and approximately 50%, between approximately 20% and approximately 50%, between approximately 25% and approximately 50%, between approximately 30% and approximately 50%, between approximately 35% and approximately 50%, between approximately 40% and approximately 50%, or between approximately 20% and approximately 40%.

A method of treating a bacterial or fungal nail infection may include topically applying any topical composition disclosed herein. For example a topical composition may be administered in a footbath or via irrigation to bath or irrigate an affected nail and/or skin surface. In some embodiments, topical administration to the affected nail and/or skin surface may include submerging the surface within the topical composition. The topical composition may be administered 1 to 4 times daily or as otherwise directed to treat the infection.

A method of treating a bacterial or fungal nail infection may include topically administering a combination of compositions selected from one or more topical nail compositions disclosed herein and/or one or more topical compositions disclosed herein. In some embodiments, a method of treating a bacterial or fungal nail infection may include topically applying a combination of topical nail compositions disclosed herein and/or a combination of topical compositions disclosed herein. The compositions may be applied sequentially or in various orders as disclosed herein.

This specification has been written with reference to various non-limiting and non-exhaustive embodiments. However, it will be recognized by persons having ordinary skill in the art that various substitutions, modifications, or combinations of any of the disclosed embodiments (or portions thereof) may be made within the scope of this specification. Thus, it is contemplated and understood that this specification supports additional embodiments not expressly set forth in this specification. Such embodiments may be obtained, for example, by combining, modifying, or reorganizing any of the disclosed steps, components, elements, features, aspects, characteristics, limitations, and the like, of the various non-limiting and non-exhaustive embodiments described in this specification.

The grammatical articles “one”, “a”, “an”, and “the”, as used in this specification, are intended to include “at least one” or “one or more”, unless otherwise indicated. Thus, the articles are used in this specification to refer to one or more than one (i.e., to “at least one”) of the grammatical objects of the article. By way of example, “a component” means one or more components, and thus, possibly, more than one component is contemplated and may be employed or used in an application of the described embodiments. Further, the use of a singular noun includes the plural, and the use of a plural noun includes the singular, unless the context of the usage requires otherwise. Additionally, the grammatical conjunctions “and” and “or” are used herein according to accepted usage. By way of example, “x and y” refers to “x” and “y”. On the other hand, “x or y” refers to “x”, “y”, or both “x” and “y”, whereas “either x or y” refers to exclusivity.

Any numerical range recited herein includes all values and ranges from the lower value to the upper value. For example, if a concentration range is stated as 1% to 50%, it is intended that values such as 2% to 40%, 10% to 30%, 1% to 3%, or 2%, 25%, 39% and the like, are expressly enumerated in this specification. These are only examples of what is specifically intended, and all possible combinations of numerical values and ranges between and including the lowest value and the highest value enumerated are to be considered to be expressly stated in this application. Numbers modified by the term “approximately” are intended to include +/−10% of the number modified.

The present disclosure may be embodied in other forms without departing from the spirit or essential attributes thereof and, accordingly, reference should be had to the following claims rather than the foregoing specification as indicating the scope of the invention. Further, the illustrations of arrangements described herein are intended to provide a general understanding of the various embodiments, and they are not intended to serve as a complete description. Many other arrangements will be apparent to those of skill in the art upon reviewing the above description. Other arrangements may be utilized and derived therefrom, such that logical substitutions and changes may be made without departing from the scope of this disclosure.

What is claimed is:

1. A combination treatment method for treating a bacterial or fungal nail infection, the method comprising:
providing one or more first composition components for mixing with diluent to formulate a first topical nail composition comprising a nail lacquer for application to an infected nail in a thin layer, wherein the one or more first composition components comprise mupirocin; and
providing one or more second composition components for mixing with diluent to formulate a second topical nail composition comprising a nail lacquer for application to the infected nail in a layer, wherein the one or more second composition components comprise tetracycline, nitrofurantoin, or combination thereof.

2. The method of claim 1, wherein the one or more first composition components further comprise itraconazole and the one or more second composition components comprise tetracycline.

3. The method of claim 1, wherein the one or more first composition components further comprise doxycycline and clotrimazole and the one or more second composition components comprise nitrofurantoin.

4. The method of claim 1, further comprising providing a third topical nail composition for application to the infected nail in a thin layer prior to application of the first topical nail composition and/or the second topical nail composition, the third topical nail composition comprising a solution including a NSAID and DMSO.

5. The method of claim 4, wherein the NSAID comprises diclofenac.

6. The method of claim 5, wherein the third topical nail composition comprises approximately 0.5% to approximately 5% diclofenac by weight and approximately 25% to approximately 50% DMSO by weight.

7. The method of claim 1, further comprising formulating the first topical nail composition comprising mixing diluent with the one or more first composition components and/or formulating the second topical nail composition comprising mixing diluent with the one or more second composition components.

8. The method of claim 7, wherein the one or more first composition components further comprise itraconazole, and wherein formulating the first topical nail composition comprises mixing approximately 10 mg to approximately 50 mg mupirocin and approximately 20 mg to approximately 100 mg itraconazole per 15 ml diluent; and wherein formulating the second topical nail composition comprises mixing approximately 500 mg to approximately 1000 mg tetracycline per 15 ml diluent.

9. The method of claim 7, wherein the one or more first composition components further comprise doxycycline and clotrimazole, and wherein formulating the first topical nail composition comprises mixing approximately 50 mg to approximately 200 mg doxycycline, approximately 10 mg to approximately 50 mg mupirocin, and approximately 15 mg to approximately 50 mg clotrimazole per 15 ml diluent, and wherein formulating the second topical nail composition comprises mixing approximately 10 mg to approximately 100 mg nitrofurantoin per 15 ml of diluent.

10. The method of claim 9, further comprising applying the first topical nail composition to infected nails in a first daily administration and the second topical nail composition to infected nails in a separate, second daily administration.

11. A combination treatment method for treating a bacterial or fungal nail infection, the method comprising:
providing first composition components for mixing with diluent to formulate a first topical nail composition comprising a nail lacquer for application to an infected nail in a thin layer, wherein the first composition components comprise:
mupirocin,
itraconazole,
urea, and
tobramycin, colistimethate, gentamycin, or combination thereof;
providing second composition components for mixing with diluent to formulate a second topical nail composition comprising a nail lacquer for application to the infected nail in a thin layer, wherein the second composition components comprise:
urea, and
one or more of voriconazole or itraconazole.

12. The method of claim 11, further comprising providing a third topical nail composition for application to the infected nail in a thin layer prior to application of the first topical nail composition and/or the second topical nail composition, the third topical nail composition comprising a solution including a NSAID and DMSO.

13. The method of claim 12, wherein the NSAID comprises diclofenac.

14. The method of claim 13, wherein the third topical nail composition comprises approximately 0.05% to approximately 5% diclofenac by weight and approximately 25% to approximately 50% DMSO by weight.

15. The method of claim 11, further comprising formulating the first topical nail composition comprising mixing diluent with the first composition components and/or formulating the second topical nail composition comprising mixing diluent with the second composition components.

16. The method of claim 15, wherein formulating the first topical nail composition comprises mixing approximately 0.6 g to approximately 1.8 g tobramycin, approximately 10 mg to approximately 50 mg mupirocin, approximately 20 mg to approximately 100 mg itraconazole, and approximately 100 mg to approximately 1000 mg urea per 15 ml diluent; and wherein formulating the second topical nail composition comprises mixing approximately 100 mg to approximately 1000 mg urea and at least one of approximately 100 mg to approximately 300 mg voriconazole or approximately 50 mg to approximately 200 mg itraconazole per 15 ml diluent.

17. The method of claim 15, wherein formulating the first topical nail composition comprises mixing approximately 40 mg to approximately 120 mg gentamicin, approximately 10 mg to approximately 50 mg mupirocin, approximately 20 mg to approximately 100 mg itraconazole, and approximately 100 mg to approximately 1000 mg urea per 15 ml diluent; and wherein formulating the second topical nail composition comprises mixing approximately 100 mg to approximately 1000 mg urea and at least one of approximately 100 mg to approximately 300 mg voriconazole or approximately 50 mg to approximately 200 mg itraconazole per 15 ml diluent.

18. The method of claim 15, wherein formulating the first topical nail composition comprises mixing approximately 100 mg to approximately 400 mg colistimethate, approximately 10 mg to approximately 50 mg mupirocin, approximately 20 mg to approximately 100 mg itraconazole, and approximately 100 mg to approximately 1000 mg urea per 15 ml diluent; and wherein formulating the second topical nail composition comprises mixing approximately 100 mg to approximately 1000 mg urea and at least one of approximately 100 mg to approximately 300 mg voriconazole or approximately 50 mg to approximately 200 mg itraconazole per 15 ml diluent.

19. The method of claim 18, further comprising applying the first topical nail composition to the infected nail in a first daily administration and the second topical nail composition to the infected nail in a separate, second daily administration.

20. A combination treatment method for treating a bacterial or fungal nail infection, the method comprising:
providing first composition components for mixing with diluent to formulate a first topical composition comprising a solution for administration to an infected nail in a footbath or via irrigation, the first composition components comprising mupirocin; and
providing one or more second composition components for mixing with diluent to formulate a second topical composition comprising a solution for administration to the infected nail in a footbath or via irrigation, the one or more second composition components comprising one or more of clindamycin, colistimethate, tetracycline, or nitrofurantoin.

21. The method of claim 20, wherein the one or more first composition components comprise mupirocin, doxycycline, and clotrimazole, and wherein the one or more second composition components comprise clindamycin, colistimethate, or both.

22. The method of claim 20, wherein the one or more second composition components comprise at least tetracycline, nitrofurantoin, or both tetracycline and nitrofurantoin.
